(19) United States
(12) Patent Application Publication
(10) Pub. No.: US 2010/0173367 A1
(43) Pub. Date: Jul. 8, 2010

(54) FUNCTIONALIZED MOLECULES COMPRISING AN AUTOSILIFICATION MOIETY AND METHODS OF MAKING AND USING SAME

(76) Inventors: Wesley D. Marner, II, Fremont, CA (US); Afshan S. Shaikh, Berkeley, CA (US); Jay D. Keasling, Berkeley, CA (US); William J. Holtz, San Francisco, CA (US); Sydnor T. Withers, III, Berkeley, CA (US); Jeffery L. Kizer, San Francisco, CA (US)

Correspondence Address: UC Berkeley - OTL;Bozicevic, Field & Francis LLP, 1900 University Avenue, Suite 200, East Palo Alto, CA, 94303, US

(21) Appl. No.: 12/441,354

(22) PCT Filed: Oct. 26, 2007
(86) PCT No.: PCT/US07/22646

§ 371 Date: Mar. 18, 2010

Related U.S. Application Data
(60) Provisional application No. 60/855,022, filed on Oct. 27, 2006.

(51) Int. Cl. C12P 5/02; C07H 21/00; C12P 13/04; C07K 1/14; C07H 99/00; C07K 16/00; C12N 9/96; C12M 1/40

(52) U.S. Cl. 435/106; 536/23.4; 536/23.2; 435/167; 530/345; 536/23.1; 536/123.1; 530/391.1; 435/188; 435/289.1

(57) ABSTRACT

The present invention provides functionalized molecules comprising a covalently linked autosilification moiety; and methods for making and using the functionalized molecules. The present invention provides nucleic acids comprising nucleotide sequence encoding polypeptides comprising an autosilification moiety. The present invention further provides silica matrices comprising a subject functionalized molecule, as well as systems and kits comprising the silica matrices. The subject functionalized molecules find use in various applications, which are also provided.

### CROSS-REFERENCE

This application claims the benefit of U.S. Provisional Patent Application No. 60/855,022, filed Oct. 27, 2006, which application is incorporated herein by reference in its entirety.

### STATEMENT REGARDING FEDERALLY SPONSORED RESEARCH

The U.S. government may have certain rights in this invention, pursuant to grant no. EEC-0425914 awarded by the National Science Foundation.

### BACKGROUND

A large number of the silica matrices produced in nature come from the marine unicellular algae known as diatoms, which are capable of producing intricate silica structures under relatively mild conditions. For example, the diatom Cylindrotheca fusiformis controls the low temperature and pressure assembly of silica using a class of peptides known as silaffins.

Biological molecules, such as proteins, have been immobilized in silica matrices, for use in various applications such as for use in catalysis, as biosensors, and the like. Silicates such as sol-gel composites or mesoporous silica, have been used to immobilize various biological molecules. Current methods for the immobilization of biological molecules in a silica support are based on simple physical entrapment of the biomolecules within the matrix. The matrix itself can result from the slow polymerization of a hydroxyl derivative of an alkoxysilane precursor, or can be formed quickly by adding a silaffin to the encapsulation mixture. Such methods rely on random entrapment events during polymerization.

The present invention provides functionalized molecules (including biological molecules and non-biological molecules) that comprise a covalently linked moiety that provides for autosilification of the functionalized molecule; and methods of making and using the functionalized molecules.

### Literature

U.S. Patent Publication No. 2005/0095690; Naik et al. ((2003) Chem. Commun. 238-239; Naik et al. (2002) J. Nanosci. Nanotechnol. 2:95-100; Kröger et al. (1999) Science 286:1129 1132; Kröger et al. (2000) Proc. Natl. Acad. Sci. USA 97:14133 14138; Brott et al. (2001) Nature 413:291 293; Kröger et al. (2001) J. Biol. Chem. 276:26066; Goeden-Wood et al. (2002) Biomacromolecules 3:874-879.

### SUMMARY OF THE INVENTION

The present invention provides functionalized molecules comprising a covalently linked autosilification moiety; and methods for making and using the functionalized molecules. The present invention provides nucleic acids comprising nucleotide sequence encoding polypeptides comprising an autosilification moiety. The present invention further provides silica matrices comprising a subject functionalized molecule, as well as systems and kits comprising the silica matrices. The subject functionalized molecules find use in various applications, which are also provided.

It has been found that covalent linkage of an autosilification moiety to a molecule provides for immobilization of the molecule in a silica matrix, without substantially adversely affecting the functional or morphological characteristics of the molecule. A molecule that comprises an autosilification moiety that is covalently linked, either directly or indirectly, to the molecule, is referred to herein as a “functionalized molecule.” A molecule that does not have a covalently linked autosilification moiety, and that is to be functionalized with an autosilification moiety, is referred to herein as a “parent molecule” or an “unmodified molecule” or a “non-functionalized molecule” or a “parent molecule of interest.” The present invention provides functionalized molecules, including functionalized macromolecules and functionalized small molecules.

The present invention provides methods of making a subject functionalized molecule. In one aspect, the methods involve covalently linking an autosilification moiety, directly or indirectly, to a parent molecule in a cell-free in vitro reaction. Any of a wide variety of parent molecules can be functionalized with an autosilification moiety, including macromolecules (both biological and non-biological) and small molecules.

In some embodiments, the parent molecule is a polypeptide. In some embodiments, where the functionalized molecule is a polypeptide comprising an autosilification moiety, the functionalized polypeptide is generated using recombinant methods. For example, a nucleic acid comprising a first nucleotide sequence encoding a parent polypeptide and a second nucleotide sequence in frame with the first nucleotide sequence and encoding an autosilification polypeptide is used to genetically modify a host cell, and the genetically modified host cell produces the functionalized polypeptide.

Functionalized molecules comprising an autosilification moiety become immobilized in a silica matrix upon reaction with a silicic acid in an appropriate buffer. The present invention further provides a silica matrix comprising a subject functionalized molecule. A subject silica matrix can be of any of a variety of forms, including, e.g., spheres, sheets, fibrils, etc. The form of the matrix will depend in part on the functionalized molecule immobilized therein.

A subject silica matrix finds use in a variety of applications, which are also provided by the present invention. In some embodiments, where the functionalized molecule comprises an enzyme, a subject matrix is useful as an in vitro, cell-free catalytic system, e.g., for generating a product of interest. In other aspects, a subject matrix is useful as a sensor, e.g., in detection of an analyte in a sample. In other aspects, a subject matrix is useful for purification of a functionalized molecule immobilized therein. In other embodiments, a functionalized molecule immobilized in a subject matrix is useful for purification of a specific binding partner of the functionalized molecule. In other embodiments, a subject matrix is useful in various diagnostic methods. In other embodiments, a subject matrix is useful in various screening methods.

### Features of the Invention

The present invention features a functionalized molecule comprising a parent molecule; and an autosilification moiety, where the autosilification moiety is covalently linked to the parent molecule. Suitable parent molecules include, e.g., a polypeptide, a nucleic acid, a lipid, a polysaccharide, an antigen, an antibody, and enzyme and a drug. Thus, in some embodiments, the parent molecule is a polypeptide, a nucleic acid, a lipid, a polysaccharide, an antigen, an antibody, or a drug. In some embodiments, the parent molecule is an enzyme. In some embodiments, the parent molecule is an antibody.

The present invention features a method of making a silica matrix comprising a molecule immobilized within the matrix. The method generally involves: contacting a subject functionalized molecule with silicic acid in the presence of a buffer, where the functionalized molecule comprises: a) a parent molecule; and b) an autosilification moiety, where the autosilification moiety is covalently linked to the parent molecule. The contacting results in binding of silica to the autosilification moiety, and immobilization of the functionalized molecule in a silica matrix. In some embodiments, the contacting is carried out at a temperature in the range of from about 0° C. to about 98° C.

The present invention features a silica matrix comprising a functionalized molecule immobilized therein, where the functionalized molecule comprises a parent molecule; and an autosilification moiety, and where the autosilification moiety is covalently linked to the parent molecule. In some embodiments, the matrix is in the form of spheres. In some of these embodiments, the spheres have an average diameter of from about 10 nm to about 1000 nm. In other embodiments, the matrix is in the form of a sheet. In other embodiments, the matrix is in the form of fibrils. In other embodiments, the matrix is immobilized in a column.

The present invention features a nucleic acid comprising a nucleotide sequence encoding a fusion polypeptide, where the fusion polypeptide comprises a parent polypeptide fused in-frame to an autosilification polypeptide. In some embodiments, the parent polypeptide is an enzyme, an antibody, a structural protein, a transmembrane protein, or a synthetic protein. In some embodiments, the nucleotide sequence is operably linked to a promoter. In some of these embodiments, the promoter is a constitutive promoter or an inducible promoter.

The present invention features an expression vector comprising a subject nucleic acid, e.g., a nucleic acid comprising a nucleotide sequence encoding a fusion polypeptide, where the fusion polypeptide comprises a parent polypeptide fused in-frame to an autosilification polypeptide. The present invention features a genetically modified host cell comprising a subject expression vector. In some embodiments, the genetically modified host cell is a eukaryotic cell. In other embodiments, the genetically modified host cell is a prokaryotic cell.

The present invention features a method of making a subject functionalized polypeptide. The method generally involves culturing a subject genetically modified host cell in a suitable medium and under conditions that permit synthesis of the encoded functionalized polypeptide by the host cell. In some embodiments, the method further involves recovering the functionalized polypeptide.

The present invention features a method of making a subject functionalized molecule. The method generally involves contacting an autosilification moiety with a molecule, where the autosilification moiety comprises a functionality that provides for covalent linkage to the molecule.

The present invention features a method of producing a product of interest. The method generally involves contacting a silica matrix with a substrate for an enzyme, wherein the silica matrix comprises a functionalized enzyme immobilized therein, wherein the functionalized enzyme comprises the enzyme; and an autosilification moiety, wherein the autosilification moiety is covalently linked to the enzyme, wherein the functionalized enzyme modifies the substrate and catalyzes production of a product, wherein the product is produced. In some embodiments, the method further comprises recovering the product. In some embodiments, the silica matrix comprises two or more functionalized enzymes in a biosynthetic pathway. In some embodiments, the product is selected from an isoprenoid, a polyketide, a macrolide, an amino acid, an alkaloid, a synthetic polymer, an antimicrobial agent, and a cancer chemotherapeutic agent.

The present invention features a method of isolating a compound from a sample. The method generally involves a) contacting a silica matrix with the sample, wherein the silica matrix comprises a functionalized first member of a specific binding pair immobilized therein, wherein the functionalized first member comprises the first member; and an autosilification moiety, wherein the autosilification moiety is covalently linked to the first member, wherein the compound is a second member of the specific binding pair that binds specifically to the first member, and wherein said contacting generates a second member-bound silica matrix; and b) removing the second member-bound silica matrix from the sample.

### BRIEF DESCRIPTION OF THE DRAWINGS

FIG. 1 depicts a scanning electron microscope (SEM) image of precipitated silica from an R5 peptide (upper panel) and precipitated silica from an R5-EAK₁ fusion.

FIG. 2 depicts an SEM image of precipitated silica from R5 fusion protein produced using a pET30 expression plasmid.

FIG. 3 depicts SEM images of R5-EAK₁ silica matrices formed in various solvent concentrations.

FIG. 4 depicts SEM images of R5-EAK₁ silica matrices formed at different temperatures.

FIG. 5 depicts SEM images of R5-EAK₁ silica matrices formed with different reaction sequences.

FIG. 6 depicts a gel showing purification of a GFP-R5 fusion protein.

FIG. 7 depicts SEM images of a GFP-R5 fusion protein.

FIG. 8 depicts the rate of p-nitrophenol production versus substrate concentration for phosphodiesterase (PDE), R5(1)-PDE, and encapsulated R5(1)-PDE.

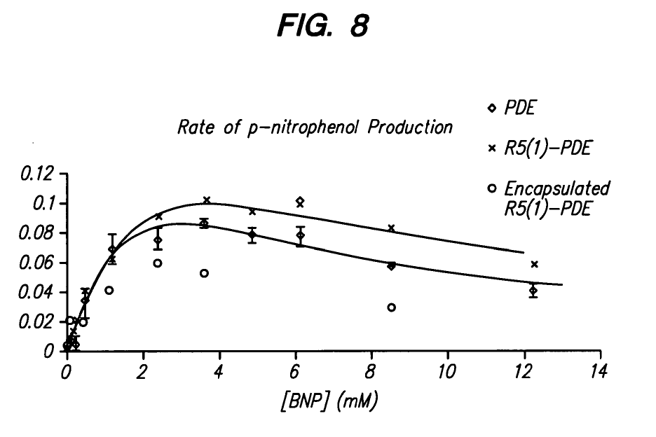

### DEFINITIONS

As used herein, the term “autosilification moiety” refers to a moiety that induces association of a subject functionalized molecule with a silica matrix. An autosilification moiety that is suitable for use herein is one that does not substantially adversely affect one or more functional and/or morphological characteristics of the parent molecule to which the autosilification moiety is covalently linked.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the phrase “specifically binds” refers to the situation in which one molecule recognizes and adheres to a particular second molecule in a sample, but does not substantially recognize or adhere to other molecules in the sample. For example, an antibody that “specifically binds” a selected antigen is one that binds the antigen with a binding affinity greater than about 10⁻⁷ M, e.g., binds with a binding affinity of at least about 10⁻⁷ M, at least about 10⁻⁸ M, or at least about 10⁻⁹ M, or greater than 10⁻⁹ M.

The term “sample” as used herein relates to a material or mixture of materials, typically, although not necessarily, in fluid form, e.g., aqueous, containing one or more components of interest. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components).

Components in a sample are termed “analytes” herein. In certain embodiments, the sample is a complex sample containing at least about 10², 5×10², 10³, 5×10³, 10⁴, 5×10⁴, 10⁵, 5×10⁵, 10⁶, 5×10⁶, 10⁷, 5×10⁷, 10⁸, 10⁹, 10¹⁰, 10¹¹, 10¹² or more species of analyte.

The term “analyte” is used herein to refer to a known or unknown component of a sample, which will specifically bind to a capture agent present in a subject silica matrix if the analyte and the capture agent are members of a specific binding pair.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides or fragments of single stranded or double stranded DNA may employed. Capture agents usually “specifically bind” one or more analytes. For example, antibodies and peptides are types of capture agents.

Accordingly, the term “capture agent” refers to a molecule or a multi-molecular complex which can specifically bind an analyte, e.g., specifically bind an analyte for the capture agent, with a dissociation constant (K_{D}) of less than about 10⁻⁶ M without binding to other targets.

The term “capture agent/analyte complex” is a complex that results from the specific binding of a capture agent with an analyte, i.e., a “binding partner pair”. A capture agent and an analyte for the capture agent specifically bind to each other under “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens, are well known in the art (see, e.g., Harlow and Lane (Antibodies: A Laboratory Manual Cold Spring Harbor Laboratory, Cold Spring Harbor, N.Y. (1989)). Conditions suitable for specific binding typically permit capture agents and target pairs that have a dissociation constant (K_{D}) of less than about 10⁻⁶ M to bind to each other, but not with other capture agents or targets.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides or polypeptides. The term “biological sample” encompasses a clinical sample, and also includes cells in culture, cell supernatants, cell lysates, serum, plasma, biological fluid, and tissue samples. In some embodiments, a biological sample will include cells.

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)). Monoclonal antibodies and “phage display” antibodies are well known in the art and encompassed by the term “antibodies”.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant” polypeptide refers to a polypeptide which is not naturally occurring, e.g., is made by the artificial combination of two otherwise separated segments of amino sequence through human intervention. Thus, e.g., a polypeptide that comprises a heterologous amino acid sequence is recombinant.

By “construct” or “vector” is meant a recombinant nucleic acid, generally recombinant DNA, which has been generated for the purpose of the expression and/or propagation of a specific nucleotide sequence(s), or is to be used in the construction of other recombinant nucleotide sequences.

The term “transformation” is used interchangeably herein with “genetic modification” and refers to a permanent or transient genetic change induced in a cell following introduction of new nucleic acid (i.e., DNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

“Operably linked” refers to a juxtaposition wherein the components so described are in a relationship permitting them to function in their intended manner. For instance, a promoter is operably linked to a coding sequence if the promoter affects its transcription or expression.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector that comprises a nucleotide sequence encoding one or more biosynthetic pathway gene products), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector that comprises a nucleotide sequence encoding a subject fusion protein), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject prokaryotic host cell is a genetically modified prokaryotic host cell (e.g., a bacterium), by virtue of introduction into a suitable prokaryotic host cell a heterologous nucleic acid, e.g., an exogenous nucleic acid that is foreign to (not normally found in nature in) the prokaryotic host cell, or a recombinant nucleic acid that is not normally found in the prokaryotic host cell; and a subject eukaryotic host cell is a genetically modified eukaryotic host cell, by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

“Isolated” or “purified” generally refers to isolation of a substance (product, compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying compounds of interest are well-known in the art and include, for example, ion-exchange chromatography, affinity chromatography and sedimentation according to density. Generally, a substance is purified when it exists in a sample in an amount, relative to other components of the sample, that is not found naturally.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

The term “array” encompasses the term “microarray” and refers to an array of functionalized molecules (e.g., capture agents for binding to aqueous analytes; enzymes for modification of a substrate; enzymes in a biosynthetic pathway; and the like).

An “array,” includes any two-dimensional or substantially two-dimensional (as well as a three-dimensional) arrangement of spatially addressable regions (i.e., “features”) containing functionalized molecules.

Any given silica matrix may carry one, two, four or more arrays. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any functionalized molecules. It will be appreciated though, that the inter-feature areas, when present, could be of various sizes and configurations. The term “array” encompasses the term “microarray” and refers to any one-dimensional, two-dimensional or substantially two-dimensional (as well as a three-dimensional) arrangement of spatially addressable regions, bearing functionalized molecules as described herein.

Each array may cover an area of less than 200 cm², or even less than 50 cm², 5 cm², 1 cm², 0.5 cm², or 0.1 cm². In certain embodiments, the silica matrix carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

An array may be spatially addressable or optically addressable. An array is “spatially addressable” when it has multiple regions of different functionalized molecules such that a region (i.e., a “feature” or “spot” of the array) at a particular predetermined location (i.e., an “address”) on the array will possess a particular function (e.g., a binding function, an enzymatic function, etc.). Array features are typically, but need not be, separated by intervening spaces. An “optically addressable” array contains an aqueous population of functionalized molecules that are labeled with optically distinguishable tags. Optically addressable arrays readily adaptable to the instant silica matrices and methods are described in greater detail in U.S. Pat. Nos. 6,649,414 and 6,524,793.

The term “analyte detection moiety”, as will be described in greater detail below, is any molecule that can indicate the presence of an analyte.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a functionalized molecule” includes a plurality of such molecules and reference to “the autosilification moiety” includes reference to one or more autosilification moieties and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides functionalized molecules comprising a covalently linked autosilification moiety; and methods for making and using the functionalized molecules. The present invention provides nucleic acids comprising nucleotide sequence encoding polypeptides comprising an autosilification moiety. The subject functionalized molecules find use in various applications, which are also provided.

### Functionalized Molecules

The present invention provides functionalized molecules, including functionalized macromolecules and functionalized small molecules, which functionalized molecules comprise an autosilification moiety covalently linked, directly or indirectly, to a molecule. A molecule that does not have a covalently linked autosilification moiety, and that is to be functionalized with an autosilification moiety, is referred to herein as a “parent molecule,” an “unmodified molecule,” a “non-functionalized molecule, or a “parent molecule of interest.”

### Autosilification Moieties

A suitable autosilification moiety is a moiety that, when covalently linked to a molecule, does not substantially adversely affect one or more functional and/or morphological characteristics of the parent molecule; and that, under suitable conditions, provides for immobilization of the functionalized molecule in a silica matrix. Functional characteristics include, but are not limited to, enzymatic activity, binding activity, and the like.

Suitable autosilification moieties include polypeptides that have affinity for silica. Suitable autosilification moieties include polypeptides that precipitate silica.

In some embodiments, an autosilification moiety is a polypeptide. Autosilification polypeptides will in some embodiments be from about 10 amino acids in length to about 100 amino acids in length, e.g., from about 10 amino acids to about 12 amino acids, from about 12 amino acids to about 15 amino acids, from about 15 amino acids to about 18 amino acids, from about 18 amino acids to about 20 amino acids, from about 20 amino acids to about 22 amino acids, from about 22 amino acids to about 25 amino acids, from about 25 amino acids to about 30 amino acids, from about 30 amino acids to about 35 amino acids, from about 35 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, or from about 90 amino acids to about 100 amino acids in length.

Exemplary autosilification moieties include a polypeptide comprising one or more of the following sequences:

(SEQ ID NO: 1)
NH₂-SSKKSGSYSGSKGSKRRIL-COOH;
(SEQ ID NO: 2)
NH₂-APPGHHHWHIHH-COOH;
(SEQ ID NO: 3)
NH₂-KPSHHHHHTGAN-COOH;
(SEQ ID NO: 4)
NH₂-MSPHPHPRHHHT-COOH;
(SEQ ID NO: 5)
NH₂-MSPHHMHHSHGH-COOH;
(SEQ ID NO: 6)
NH₂-LPHHHHLHTKLP-COOH;
(SEQ ID NO: 7)
NH₂-APHHHHPHHLSR-COOH;
(SEQ ID NO: 8)
NH₂-SSKKSGSYSGSKGSKRRNL-COOH;
(SEQ ID NO: 9)
NH₂-SSKKSGSYSGYSTKKSGSRRIL-COOH;
and
(SEQ ID NO: 10)
NH₂-SSKKSGSYYSYGTKKSGSYSGYSTKKSASRRIL-COOH.

In some embodiments, an autosilification moiety will comprise a modified lysine residue. Exemplary modified lysine residues include ε-N,N-dimethyllysine, ε-N,N,N-trimethyl-δ-hydroxylysine, and a polyamine-modified lysine.

Other suitable autosilification moieties include polyallylamines such as tripropylene tetramine and pentapropylene hexamine; polyallylamine hydrochloride; polyethylene imine; poly-serine; polyamines (which may consist of N-methyl-propyleneimine repeated units attached to putrescine); poly(ethylene glycol); poly-proline; poly(L-lysine hydrobromide); poly(L-arginine hydrochloride); and cysteine-lysine block copolypeptides.

In some embodiments, an autosilification moiety comprises a poly-lysine, e.g., (K)ₙ, where n=3-20. In some embodiments, an autosilification moiety comprises a poly-arginine, e.g., (R)ₙ, where n=3-20.

### Parent Molecules

A subject functionalized molecule comprises a molecule of interest, e.g., macromolecule or a small molecule, covalently linked, directly or indirectly, to an autosilification moiety. Suitable macromolecules include, but are not limited to, naturally-occurring, synthetic, and recombinant biomolecules, including, but not limited to, polypeptides, polynucleotides, lipoproteins, glycoproteins, glycolipoproteins, polysaccharides, lipopolysaccharides, mucopolysaccharides, lipids, and the like; synthetic macromolecules, including, but not limited to, synthetic polypeptides, synthetic polynucleotides, synthetic polymers, and the like.

Suitable small molecules include, but are not limited to, amino acids, nucleotides, nucleosides, sugars, steroids, lipids, metal ions, drugs, hormones, amides, amines, carboxylic acids, vitamins and coenzymes, alcohols, aldehydes, ketones, fatty acids, porphyrins, carotenoids, plant growth regulators, phosphate esters and nucleoside diphospho-sugars, synthetic small molecules such as pharmaceutically or therapeutically effective agents, monomers, peptide analogs, haptens, steroid analogs, inhibitors, mutagens, carcinogens, antimitotic drugs, antibiotics, ionophores, antimetabolites, amino acid analogs, antibacterial agents, transport inhibitors, surface-active agents (surfactants), mitochondrial and chloroplast function inhibitors, electron donors, carriers and acceptors, synthetic substrates for proteases, substrates for phosphatases, substrates for esterases and lipases and protein modification reagents.

Suitable proteins include, for example, immunoglobulins, cytokines, enzymes, hormones, cancer antigens, nutritional markers, tissue specific antigens, etc. Suitable proteins include, by way of illustration and not limitation, protamines, histones, albumins, globulins, scleroproteins, phosphoproteins, mucoproteins, chromoproteins, lipoproteins, nucleoproteins, glycoproteins, T-cell receptors, proteoglycans, histocompatibility antigens, somatotropin, prolactin, insulin, pepsin, proteins found in human plasma, blood clotting factors, protein hormones such as, e.g., follicle-stimulating hormone, luteinizing hormone, luteotropin, prolactin, chorionic gonadotropin, tissue hormones, cytokines, cancer antigens such as, e.g., PSA, CEA, a-fetoprotein, acid phosphatase, CA19.9 and CA125, tissue specific antigens, such as, e.g., alkaline phosphatase, myoglobin, CPK-MB and calcitonin, and peptide hormones.

Suitable polymers include, but are not limited to, polyalkylenes, polyamides, poly(meth)acrylates, polysulfones, polystyrenes, polyethers, polyvinyl ethers, polyvinyl esters, polycarbonates, polyvinyl halides, polysiloxanes, POMA, PEG, and copolymers of any two or more of the foregoing. Suitable biocompatible and/or biodegradable synthetic polymers include, but are not limited to, polystyrenes, e.g., Poly(styrene-co-chloromethylsytrene), Poly(styrene-co-chloromethylstyrene-co-methyl-4-vinylbenzyl)ether, Poly(styrene-co-chloromethylsytrene); polyphosphoesters, e.g., Poly[1,4-bis(hydroxyethyl)terephthalate-co-ethyloxyphosphate]; aliphatic polyesters, e.g., Poly(1,4-butylene adipate-co-polycaprolactam), Polycaprolactone; polyglycolide, Poly(DL-lactide), Poly(DL-lactide-co-caprolactone)DL-lactide, Poly(L-lactide-co-caprolactone-co-glycolide), Poly(DL-lactide-co-glycolide), Poly[(R)-3-hydroxybutyric acid], Poly(3-hydroxybutyric acid-co-3-hydroxyvaleric acid), and Poly(1,4-butylene succinate); modified polysaccharides, e.g., (Acrylamidomethyl)cellulose acetate butyrate, (Acrylamidomethyl)cellulose acetate propionate, Cellulose acetate, Cellulose acetate butyrate, Cellulose acetate phthalate, Cellulose acetate propionate, Cellulose acetate trimellitate, Cellulose nitrate, starch, chitosan, Dextrin palmitate, Ethyl cellulose, 2-Hydroxyethyl cellulose, Hydroxyethylcellulose ethoxylate, Hydroxypropyl cellulose, (Hydroxypropyl)methyl cellulose, Hydroxypropyl methyl cellulose phthalate, Maltodextrin, methylcellulose, Methyl 2-hydroxyethyl cellulose, and Sodium carboxymethyl cellulose; poly(ethylene glycol) (PEG)-based polymers, e.g., Poly(ethylene glycol)-block-polylactide methyl ether, Di[poly(ethylene glycol)]adipate, Hexaethylene glycol, Pentaethylene glycol, Polyethylene-block-poly(ethylene glycol), Poly(ethylene glycol), Poly(ethylene glycol)dibenzoate, Poly(ethylene glycol)bis(carboxymethyl)ether, Poly(ethylene glycol)butyl ether, Poly(ethylene glycol)diacrylate, Poly(ethylene glycol)dimethacrylate, Polyethylene glycol dimethyl ether, Polyethylene glycol distearate, Poly(ethylene glycol)ethyl ether methacrylate, Poly(ethylene glycol)methacrylate, Poly(ethylene glycol)-block-poly(ε-caprolactone)methyl ether, Poly(ethylene oxide), Poly(ethylene oxide)-block-polycaprolactone, Poly(ethylene oxide)-block-polylactide, Tetraethylene glycol dimethyl ether, and Poly(ethylene glycol) di-(4-hydroxyphenyl)diphenylphosphine; PEG-PPG copolymers, e.g., Poly(ethylene glycol)-block-poly(propylene glycol)-block-poly(ethylene glycol), Poly(propylene glycol)-block-poly(ethylene glycol)-block-poly(propylene glycol) bis(2-aminopropyl ether); polyanhydrides, e.g., 1,3-Bis(4-carboxyphenoxy)propane, 1,6-Bis(p-acetoxycarbonylphenoxy)hexane, and Poly[1,6-bis(p-carboxyphenoxy)hexane]; PVA and copolymers, e.g., Poly(vinyl alcohol), Poly(ethylene glycol)-block-polypropylene glycol)-block-poly(ethylene glycol), Poly(vinyl alcohol-co-ethylene) ethylene, Poly(vinyl alcohol-co-vinyl acetate-co-itaconic acid), and Poly(vinyl chloride-co-vinyl acetate-co-vinyl alcohol); hydrogels, e.g., Poly(acrylic acid-co-acrylamide) Potassium salt cross-linked, Poly(2-hydroxyethyl methacrylate), Poly(isobutylene-co-maleic acid), and Poly(N-isopropylacrylamide); polycationic polymers, e.g., polyalkylamine (PAM), polyethylenimine (PE), polylysine (PL).

Suitable drugs include, but are not limited to, pharmaceutically active compounds, metabolites, and the like. Included among drugs of interest are the alkaloids. Among the alkaloids are morphine alkaloids, which includes morphine, codeine, heroin, dextromethorphan, their derivatives and metabolites; cocaine alkaloids, which include cocaine and benzoylecgonine, their derivatives and metabolites; ergot alkaloids, which include the diethylamide of lysergic acid; steroid alkaloids; iminazoyl alkaloids; quinazoline alkaloids; isoquinoline alkaloids; quinoline alkaloids, which include quinine and quinidine; diterpene alkaloids, their derivatives and metabolites; steroids, which includes the estrogens, androgens, andreocortical steroids, bile acids, cardiotonic glycosides and aglycones, which includes digoxin and digoxigenin, saponins and sapogenins, their derivatives and metabolites, and steroid mimetic substances, such as diethylstilbestrol; lactams having from 5 to 6 annular members, which include the barbituates, e.g., phenobarbital and secobarbital, diphenylhydantoin, primidone, ethosuximide, and their metabolites; aminoalkylbenzenes, with alkyl of from 2 to 3 carbon atoms, which includes the amphetamines; catecholamines, which includes ephedrine, L-dopa, epinephrine; narceine; papaverine; and metabolites of the foregoing; benzheterocyclics, which include oxazepam, chlorpromazine, tegretol, their derivatives and metabolites, the heterocyclic rings being azepines, diazepines and phenothiazines; purines, which includes theophylline, caffeine, their metabolites and derivatives; hormones such as thyroxine, cortisol, triiodothyronine, testosterone, estradiol, estrone, progestrone, polypeptides such as angiotensin, LHRH, and immunosuppressants such as cyclosporin, FK506, mycophenolic acid, and so forth; vitamins such as A, B, e.g. B12, C, D, E and K, folic acid, thiamine; prostaglandins, which differ by the degree and sites of hydroxylation and unsaturation; tricyclic antidepressants, which include imipramine, dismethylimipramine, amitriptyline, nortriptyline, protriptyline, trimipramine, chlomipramine, doxepine, and desmethyldoxepin; anti-neoplastics, which include methotrexate; antibiotics, which include penicillin, chloromycetin, actinomycetin, tetracycline, terramycin, the metabolites and derivatives; the nucleosides and nucleotides, which include ATP, NAD, FMN, adenosine, guanosine, thymidine, and cytidine with their appropriate sugar and phosphate substituents; methadone, meprobamate, serotonin, meperidine, lidocaine, procainamide, acetylprocainamide, propranolol, griseofulvin, valproic acid, butyrophenones, antihistamines, chloramphenicol, anticholinergic drugs, such as atropine, their metabolites and derivatives; aminoglycosides, such as gentamicin, kanamicin, tobramycin, and amikacin.

Suitable drugs also include cancer chemotherapeutic agents. Cancer chemotherapeutic agents include non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

In some embodiments, the parent molecule is an antibody that has specificity for an antigen or a hapten. Antibodies include polyclonal antibodies, monoclonal antibodies, single-chain antibodies, artificial antibodies, humanized antibodies, chimeric antibodies, and antigen-binding antibody fragments (e.g., Fv, F(ab′)₂, Fab, Fab′, scFv, etc.). Antibodies include a complete immunoglobulin or fragment thereof, which immunoglobulins include the various classes and isotypes, such as IgA, IgD, IgE, IgG1, IgG2a, IgG2b and IgG3, IgM, etc. The antibody will in some embodiments comprise a detectable label or a moiety that provides for detection using a detectably labeled specific binding partner. Suitable direct labels include, but are not limited to, radioactive labels (e.g., ¹²⁵I, etc.); enzyme labels, where the enzyme generates a product that is detectable by a colorimetric or fluorimetric assay, e.g., β-galactosidase, luciferase, horse radish peroxidase, alkaline phosphatase; fluorescent proteins, e.g. a green fluorescent protein; and the like. Indirect labels include secondary antibodies that are detectably labeled; a member of a specific binding pair (e.g., biotin/avidin, etc.) that is detectably labeled; and the like.

In some embodiments, the parent molecule comprises a targeting moiety, e.g., a moiety that provides for or mediates binding to a molecule expressed in a specific cell type or specific tissue. Suitable targeting moieties include moieties comprising a ligand, an antigen, a hapten, biotin, lectin, galactose, galactosamine, a protein, a histone, a polypeptide, a lipid, a lectin, a carbohydrate, a vitamin, or a combination of two or more of the foregoing.

In some embodiments, the parent molecule is an antigen. In some embodiments, the antigen is from an infectious agent, including protozoan, bacterial, fungal (including unicellular and multicellular), and viral infectious agents. Examples of suitable viral antigens are described herein and are known in the art. Bacteria include Hemophilus influenza, Mycobacterium tuberculosis and Bordetella pertussis. Protozoan infectious agents include malarial plasmodia, Leishmania species, Trypanosoma species and Schistosoma species. Fungi include Candida albicans.

In some embodiments, the antigen is a viral antigen. Viral polypeptide antigens include, but are not limited to, core proteins such as HIV gag proteins (including, but not limited to, membrane anchoring (MA) protein, core capsid (CA) protein and nucleocapsid (NC) protein), HIV polymerase, influenza virus matrix (M) protein and influenza virus nucleocapsid (NP) protein. References discussing influenza vaccination include Scherle and Gerhard (1988) Proc. Natl. Acad. Sci. USA 85:4446-4450; Scherle and Gerhard (1986) J. Exp. Med. 164:1114-1128; Granoff et al. (1993) Vaccine 11:S46-51; Kodihalli et al. (1997) J. Virol. 71:3391-3396; Ahmeida et al. (1993) Vaccine 11:1302-1309; Chen et al. (1999) Vaccine 17:653-659; Govorkova and Smirnov (1997) Acta Virol. (1997) 41:251-257; Koide et al. (1995) Vaccine 13:3-5; Mbawuike et al. (1994) Vaccine 12:1340-1348; Tamura et al. (1994) Vaccine 12:310-316; Tamura et al. (1992) Eur. J. Immunol. 22:477-481; Hirabayashi et al. (1990) Vaccine 8:595-599. Other examples of antigen polypeptides are group- or sub-group specific antigens, which are known for a number of infectious agents, including, but not limited to, adenovirus, herpes simplex virus, papilloma virus, respiratory syncytial virus and poxviruses.

In some embodiments, the antigen is a tumor antigen. Tumor antigens (e.g., tumor specific antigens) include, but are not limited to, such as Her-2/neu, Mart1, carcinoembryonic antigen (CEA), gangliosides, human milk fat globule (HMFG), mucin (MUC1), MAGE antigens, BAGE antigens, GAGE antigens, gp100, prostate specific antigen (PSA), and tyrosinase.

In some embodiments, the parent molecule is a fluorescent, chromogenic, or chemiluminescent protein. Suitable fluorescent proteins include, but are not limited to, a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; U.S. Patent Publication No. 2002/0197676, or U.S. Patent Publication No. 2005/0032085; a red fluorescent protein; a yellow fluorescent protein; and the like.

In some embodiments, the parent molecule is an enzyme. Suitable enzymes include, but are not limited to, a lipase, an esterase, a protease, a glycosidase, a glycosyl transferase, a phosphatase, a kinase, a lipase, a hydroxylase, an oxygenase, a polymerase (e.g., a DNA polymerase, an RNA polymerase), a peroxidase, a hydrolase, a hydratase, a nitrilase, a transaminase, an amidase, a phosphodiesterase, and an acylase. Suitable enzymes also include an enzyme that, in the presence of a suitable substrate, gives rise to a detectable signal, e.g., horse radish peroxidase, alkaline phosphatase, β-galactosidase, luciferase.

Suitable enzymes also include two or more enzymes in an anabolic or catabolic pathway. For example, suitable enzymes include enzymes that catalyze the synthesis of isoprenoid compounds via a mevalonate pathway. See, e.g., U.S. Patent Application Nos. 2003/0148479 and 2006/0079476.

In some embodiments, the parent molecule is a receptor for a ligand, e.g., a hormone receptor, a receptor that binds to a neurotransmitter (e.g., an acetylcholine receptor), and the like. In some embodiments, the parent molecule is a transmembrane protein, e.g., a G-protein coupled receptor, an ion channel, and the like.

In some embodiments, the parent molecule is a dye. Suitable dyes include, but are not limited to, fluorophores, a wide variety of which are known in the art. Exemplary fluorophores include fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1(5,5′,6,6′-tetrachloro-1,1′,3,3′-tetraethylbenzimidazoylcarbocyanine iodide), tetrabromorhodamine 123, rhodamine 6G, TMRM (tetramethylrhodamine-, methyl ester), TMRE (tetramethylrhodamine, ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-(vinylsulfonyl)phenyl]naphthalimide-3,5disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino)naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; 5-(2′-aminoethyl)aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, and fluorescent europium and terbium complexes; and the like. Fluorophores of interest are further described in WO 01/42505 and WO 01/86001.

In some embodiments, the parent molecule of interest a therapeutic protein. Suitable therapeutic proteins include, but are not limited to, an interferon (e.g., IFN-γ, IFN-α, IFN-β, IFN-ω; IFN-τ; as described in more detail below); an insulin (e.g., Novolin, Humulin, Humalog, Lantus, Ultralente, etc.); an erythropoietin (e.g., Procrit®, Eprex®, or Epogen® (epoetin-α); Aranesp® (darbepoietin-α); NeoRecormon®, Epogin® (epoetin-β); and the like); an antibody (e.g., a monoclonal antibody) (e.g., Rituxan® (rituximab); Remicade® (infliximab); Herceptin® (trastuzumab); Humira™ (adalimumab); Xolair® (omalizumab); Bexxar® (tositumomab); Raptiva™ (efalizumab); Erbitux™ (cetuximab); and the like), including an antigen-binding fragment of a monoclonal antibody; a blood factor (e.g., Activase® (alteplase) tissue plasminogen activator; NovoSeven® (recombinant human factor VIIa); Factor VIIa; Factor VIII (e.g., Kogenate®); Factor IX; β-globin; hemoglobin; and the like); a colony stimulating factor (e.g., Neupogen® (filgrastim; G-CSF); Neulasta (pegfilgrastim); granulocyte colony stimulating factor (G-CSF), granulocyte-monocyte colony stimulating factor, macrophage colony stimulating factor, megakaryocyte colony stimulating factor; and the like); a growth hormone (e.g., a somatotropin, e.g., Genotropin®, Nutropin®, Norditropin®, Saizen®, Serostim®, Humatrope®, etc.; a human growth hormone; and the like); an interleukin (e.g., IL-1, IL-2, including, e.g., Proleukin®; IL-3, IL-4, IL-5, IL-6, IL-7, IL-8, IL-9; etc.); a growth factor (e.g., Regranex® (beclapermin; PDGF); Fiblast® (trafermin; bFGF); Stemgen® (ancestim; stem cell factor); keratinocyte growth factor; an acidic fibroblast growth factor, a stem cell factor, a basic fibroblast growth factor, a hepatocyte growth factor; and the like); a soluble receptor (e.g., a TNF-α-binding soluble receptor such as Enbrel® (etanercept); a soluble vascular endothelial growth factor (VEGF) receptor; a soluble interleukin receptor; a soluble γ/δ T cell receptor; and the like); an enzyme (e.g., α-glucosidase; Cerazyme® (imiglucarase; β-glucocerebrosidase, Ceredase® (alglucerase;); an enzyme activator (e.g., tissue plasminogen activator); a chemokine (e.g., IP-10; Mig; Groa/IL-8, RANTES; MIP-1α; MIP-1β; MCP-1; PF-4; and the like); an angiogenic agent (e.g., VEGF; an anti-angiogenic agent (e.g., a soluble VEGF receptor); a protein vaccine; a neuroactive peptide such as bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, warfarin, dynorphin, neurotensin, motilin, thyrotropin, neuropeptide Y, luteinizing hormone, calcitonin, insulin, glucagon, vasopressin, angiotensin II, thyrotropin-releasing hormone, vasoactive intestinal peptide, a sleep peptide, etc.; other therapeutic proteins such as a thrombolytic agent, an atrial natriuretic peptide, bone morphogenic protein, thrombopoietin, relaxin, glial fibrillary acidic protein, follicle stimulating hormone, a human alpha-1 antitrypsin, a leukemia inhibitory factor, a transforming growth factor, a tissue factor, an insulin-like growth factor, a luteinizing hormone, a follicle stimulating hormone, a macrophage activating factor, tumor necrosis factor, a neutrophil chemotactic factor, a nerve growth factor, a tissue inhibitor of metalloproteinases; a vasoactive intestinal peptide, angiogenin, angiotropin, fibrin; hirudin; a leukemia inhibitory factor; an IL-1 receptor antagonist (e.g., Kineret® (anakinra)); and the like.

In some embodiments, the parent molecule is a synthetic polypeptide, e.g., a poly(Glu-Ala-Lys) (poly(EAK)) polypeptide. For example, in some embodiments, the parent molecule is (EAK)ₘ, where m=1 to 100, e.g., m=1, 2, 3-5, 5-10, 10-15, 15-20, 20-25, 25-30, 30-35, 35-40, 40-45, 45-50, 50-60, 60-70, 70-80, 80-90, or 90-100.

In some embodiments, the parent molecule is a self-assembling polypeptide. In some embodiments, the parent molecule is an elastin-like protein. For example, in some embodiments, the parent molecule comprises an amino acid sequence comprising the repeat unit [VPGXG]ₙ (SEQ ID NO:11), where X is any amino acid other than proline, and where n is an integer from 1 to 5, from 5 to 10, from 10 to 20, or more than 20. See, e.g., U.S. Pat. No. 6,770,442.

In other embodiments, the parent molecule comprises repeats of the amino acid sequence SGAGAG (SEQ ID NO:12; G=glycine; A=alanine; S=serine). This repeating unit is found in a naturally occurring silk fibroin protein, which can be represented as GAGAG(SGAGAG)₈SGAAGY (SEQ ID NO:13; Y=tyrosine).

Other suitable parent molecules comprising tandem or non-tandem repeat units include parent molecules that comprise one or more of the following repeat units: a) LKPNM (SEQ ID NO:14); b) KPNM (SEQ ID NO:15); c) VVYP; d) KPN; e) DKP; f) YKP; g) EKP; h) DAP; i) EAP; j) HPP; k) VPP; l) LK; m) PN; and n) NM. See, e.g., U.S. Pat. No. 7,060,467.

In some embodiments, the parent molecule is a peptide nucleic acid. Peptide nucleic acids (PNAs) are non-naturally occurring polyamides (also properly characterized as pseudopeptides) which can hybridize to nucleic acids (DNA and RNA) with sequence specificity. See, e.g., U.S. Pat. Nos. 6,770,442, 5,539,082, 5,527,675, 5,623,049, 5,714,331, 5,736,336, 5,773,571, and 5,786,571.

Suitable parent molecules also include fusion proteins, e.g., a protein that comprises a first protein fused in-frame to a heterologous fusion partner. Non-limiting examples of such fusion proteins include a fusion protein that comprises an antibody fused to a protein that generates a detectable signal, e.g, a fluorescent protein, a chromogenic protein, an enzyme that produces a fluorescent, luminescent, or chromogenic product upon action on a substrate; a Lumio™ tag (e.g., a peptide of the sequence Cys-Cys-Xaa-Xaa-Cys-Cys, where Xaa is any amino acid other than cysteine, e.g., where Xaa-Xaa is Pro-Gly, which peptide is specifically bound by a fluorescein derivative having two As(III) substituents, e.g., 4′,5′-bis(1,3,2-dithioarsolan-2-yl)fluorescein; see, e.g., Griffin et al. (1998) Science 281:269; Griffin et al. (2000) Methods Enzymol. 327:565; and Adams et al. (2002) J. Am. Chem. Soc. 124:6063); and the like.

In some embodiments, the parent molecule is a fusion protein comprising a metal-binding peptide. Suitable metal ion binding peptides include, but are not limited to, poly(histidine) peptides, e.g., (His)₆; etc. See, e.g., U.S. Pat. No. 5,284,933; U.S. Pat. No. 5,310,663; U.S. Pat. No. 4,569,794; and U.S. Pat. No. 5,594,115; and U.S. Patent Publication Nos. 2002/0164718 and 2004/0180415.

Where the parent molecule is a polypeptide, a subject functionalized molecule is a fusion protein comprising a parent polypeptide fused to an autosilification moiety. In some embodiments, a subject functionalized protein is of the formula NH₂-(A)ₙ-X—B—X-(A)_{c}-COOH, where (A)ₙ is an amino-terminal portion of the parent polypeptide of interest, X is an optional linker, B is the autosilification moiety, and (A)_{c} is a carboxyl-terminal portion of the parent polypeptide of interest. In other embodiments, the fusion protein is of the formula NH₂-A-X—B—COOH, where A is the parent polypeptide, X is an optional linker, and B is the autosilification moiety. In other embodiments, the encoded fusion protein is of the formula NH₂—B—X-A-COOH, A is the parent polypeptide, X is an optional linker, and B is the autosilification moiety.

In other embodiments, the functionalized protein is of the formula B₆-Aₘ, where B is the autosilification moiety, A is the parent polypeptide, where n=1-50, and where m=1-50. For example, in some embodiments, a subject functionalized protein is of the formula Bₙ-(EAK)ₘ, where n=1-50, and where m=1-50. As another example, in some embodiments, a subject functionalized protein comprises a repeat unit of the formula Bₙ-(VPGXG)ₘ, where X is any amino acid except proline, and where n=1-50, and where m=1-50. X can be the same or different from one repeat unit to the next. For example, a first repeat unit can comprise the sequence VPGAG (SEQ ID NO:16); a second repeat unit can comprise the sequence VPGSG (SEQ ID NO:17); a third repeat unit can comprise the sequence VPGTG (SEQ ID NO:18); and so on.

### Methods of Making a Functionalized Molecule

The instant invention provides methods of making a subject functionalized molecule.

In some embodiments, the methods involve covalently linking an autosilification moiety, directly or indirectly, to a parent molecule of interest in a cell-free in vitro reaction with a silicic acid in an appropriate buffer. In other embodiments, e.g., where the parent molecule of interest is a protein, the methods involve recombinant techniques.

### Cell-Free In Vitro Methods

In some embodiments, the methods involve covalently linking an autosilification moiety, directly or indirectly, to a parent molecule of interest in a cell-free in vitro reaction. In some embodiments, the autosilification moiety is covalently linked directly to a parent molecule. In other embodiments, the autosilification moiety is covalently linked to a parent molecule via a linker. Cell-free in vitro methods of covalently linking a molecule of interest with an autosilification moiety can employ any known chemistry for covalent linkage of two molecules.

An autosilification moiety can be covalently linked to an amine, thiol, or carboxyl group present in, or introduced into, a molecule of interest, using standard chemistry. Standard chemistries such as reaction with an N-hydroxysuccinimide ester, reaction of a maleimide with a thiol group, carbodiimide chemistry, and the like, can be used to link a molecule of interest (e.g., a protein of interest) to an autosilification moiety. An autosilification moiety and/or a parent molecule can be derivatized with one or more functional groups (e.g., acyl fluorides, anhydrides, oxiranes, aldehydes, hydrazides, acyl azides, aryl azides, diazo compounds, benzophenones, carbodiimides, imidoesters, isothiocyanates, NHS esters, CNBr, maleimides, tosylates, tresyl chloride, maleic anhydrides, and carbonyldiimidazoles) that provide for covalent linkage.

Where the molecule of interest is a nucleic acid, the autosilification moiety can be attached to the 3′-end of the nucleic acid through solid support chemistry. For example, the nucleic acid portion can be added to an autosilification moiety portion that has been pre-synthesized on a support. Haralambidis et al. (1990a) Nucleic Acids Res. 18:493-499; and Haralambidis et al. (1990b) Nucleic Acids Res. 18:501-505. Alternatively, the nucleic acid can be synthesized such that it is connected to a solid support through a cleavable linker extending from the 3′-end. Upon chemical cleavage of the nucleic acid from the support, a terminal thiol group is left at the 3′-end of the nucleic acid (Zuckermann et al. (1987) Nucleic Acids Res. 15:5305-5321; and Corey et al. (1987) Science 238:1401-1403) or a terminal amino group is left at the 3′-end of the nucleic acid (Nelson et al. (1989) Nucleic Acids Res. 17:1781-1794). The thiol-modified nucleic acid can be covalently linked to a carboxyl groups of an autosilification moiety, as described in Sinha et al. (1991), pp. 185-210, Oligonucleotide Analogues: A Practical Approach, IRL Press. Conjugation of the amino-modified nucleic acid to amino groups of an autosilification moiety can be performed as described in Benoit et al. (1987) Neuromethods 6:43-72. Coupling of a nucleic acid carrying an appended maleimide to the thiol side chain of a cysteine residue of a peptide has also been described. Tung et al. (1991) Bioconjug. Chem. 2:464-465.

In some embodiments, an autosilification moiety is covalently linked to a molecule of interest via a linker. In some embodiments, the linker is a peptide. The linker peptide may have any of a variety of amino acid sequences. Proteins can be joined by a spacer peptide, generally of a flexible nature, although other chemical linkages are not excluded. The linker may be a cleavable linker. Suitable linker sequences will generally be peptides of between about 5 and about 50 amino acids in length, or between about 6 and about 25 amino acids in length. Peptide linkers with a degree of flexibility will generally be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use according to the present invention.

Suitable linker peptides frequently include amino acid sequences rich in alanine and proline residues, which are known to impart flexibility to a protein structure. Exemplary linkers have a combination of glycine, alanine, proline and methionine residues, such as AAAGGM (SEQ ID NO:19); AAAGGMPPAAAGGM (SEQ ID NO:20); AAAGGM (SEQ ID NO:21); and PPAAAGGM (SEQ ID NO:22). Other exemplary linker peptides include IEGR (SEQ ID NO:23); and GGKGGK (SEQ ID NO:24). However, any flexible linker generally between about 5 and about 50 amino acids in length may be used. Linkers may have virtually any sequence that results in a generally flexible peptide, including alanine-proline rich sequences.

### Recombinant Methods

In some embodiments, e.g., where the parent molecule of interest is a protein, the methods involve recombinant techniques. In these embodiments, the functionalized molecule, e.g., a protein comprising a covalently linked autosilification moiety, is a fusion protein, and the method involves use of a nucleic acid comprising a nucleotide sequence encoding the fusion protein. For example, in some embodiments, the encoded fusion protein is of the formula NH₂-A-X—B—COOH, where A is the parent polypeptide, X is an optional linker, and B is the autosilification moiety. In other words, in some embodiments, the autosilification moiety is fused in-frame to the carboxyl-terminus of the parent polypeptide of interest. In other embodiments, the encoded fusion protein is of the formula NH₂—B—X-A-COOH, A is the parent polypeptide, X is an optional linker, and B is the autosilification moiety. In other words, in some embodiments, the autosilification moiety is fused in-frame to the amino-terminus of the parent polypeptide of interest. In other embodiments, the encoded fusion protein is of the formula NH₂-(A)ₙ-X—B—X-(A)_{c}-COOH, where (A)ₙ is an amino-terminal portion of the parent polypeptide of interest, X is an optional linker, B is the autosilification moiety, and (A)_{c} is a carboxyl-terminal portion of the parent polypeptide of interest. In other words, in some embodiments, the autosilification moiety is fused in-frame at a site internal to the parent polypeptide of interest. The present invention thus provides a fusion protein comprising: a) a parent polypeptide of interest; and b) an autosilification polypeptide fused in-frame with the parent polypeptide.

In these embodiments, a host cell that is genetically modified with a nucleic acid (e.g., a subject nucleic acid, as described below) comprising a nucleotide sequence encoding a subject fusion protein is cultured in vitro in a suitable medium and for such a time that the encoded fusion protein is produced by the cell. The fusion protein can be isolated from the cell culture medium and/or from a cell lysate.

In some embodiments, a subject fusion protein is isolated from cell lysate and/or cell culture medium, e.g., using standard methods, e.g., high performance liquid chromatography, size exclusion chromatography, affinity chromatography (e.g., using immobilized antibody specific for the autosilification moiety, or immobilized antibody specific for the parent protein of interest), immobilized metal ion affinity chromatography, ion-exchange chromatography, etc.

In some embodiments, a subject fusion protein comprises a metal-binding peptide; and the fusion protein is purified using immobilized metal ion affinity chromatography. Suitable metal ion binding peptides include, but are not limited to, poly(histidine) peptides, e.g., (His)₆; etc. See, e.g., U.S. Pat. No. 5,284,933; U.S. Pat. No. 5,310,663; U.S. Pat. No. 4,569,794; and U.S. Pat. No. 5,594,115; and U.S. Patent Publication Nos. 2002/0164718 and 2004/0180415.

In some embodiments, a subject fusion protein is pure, e.g., at least about 40% pure, at least about 50% pure, at least about 60% pure, at least about 70% pure, at least about 80% pure, at least about 90% pure, at least about 95% pure, at least about 98%, or more than 98% pure, where “pure” in the context of a subject fusion protein refers to a subject fusion protein that is free from other proteins, macromolecules, contaminants, etc.

### Nucleic Acids

The present invention provides recombinant nucleic acids comprising a nucleotide sequence encoding a subject fusion protein, e.g., a fusion protein comprising: a) a parent polypeptide of interest; and b) an autosilification polypeptide fused in-frame with the parent polypeptide.

Nucleotide sequences encoding a parent protein of interest can be sequences that are known in the art, or can be deduced from the amino acid sequence of the parent protein of interest.

Nucleotide sequences encoding autosilification moieties are known in the art, or can be deduced from the amino acid sequence of an autosilification moiety. For example, nucleotide sequences encoding silaffins are known in the art. See, e.g., GenBank Accession Nos.: AY706751 (Thalassiosira pseudonana silaffin precursor (Sil3) nucleotide sequence; SEQ ID NO:25); AY706750 (Thalassiosira pseudonana silaffin precursor (Sil2) nucleotide sequence; SEQ ID NO:26); AY706749 (Thalassiosira pseudonana silaffin precursor (Sil1) nucleotide sequence; SEQ ID NO:27); and AF191634 (Cylindrotheca fusiformis silaffin precursor (sil1) nucleotide sequence; SEQ ID NO:28).

In some embodiments, a nucleic acid encoding a subject fusion protein comprises a nucleotide sequence encoding an autosilification moiety, where the nucleotide sequence encoding the autosilification moiety comprises a nucleotide sequence having at least about 75%, at least about 80%, at least about 85%, at least about 90%, at least about 95%, or at least about 98%, or more, nucleotide sequence identity to the following nucleotide sequence: 5′-TCT TCC TCT AAA AAG TCT GGT TCC TAC TCT GGT AGC AAA GGC TCC AAA CGT CGC ATC CTG-3′ (SEQ ID NO:29). In some embodiments, autosilification moiety-encoding nucleotide sequence comprises the nucleotide sequence 5′-TCT TCC TCT AAA AAG TCT GGT TCC TAC TCT GGT AGC AAA GGC TCC AAA CGT CGC ATC CTG-3′ (SEQ ID NO:29).

In some embodiments, the encoded subject fusion protein will comprise a non-native amino acid sequence that provides for secretion of the fusion protein from the cell. Those skilled in the art are aware of such secretion signal sequences. Secretion signals that are suitable for use in bacteria include, but are not limited to, the secretion signal of Braun's lipoprotein of E. coli, S. marcescens, E. amylosora, M. morganii, and P. mirabilis, the TraT protein of E. coli and Salmonella; the penicillinase (PenP) protein of B. licheniformis and B. cereus and S. aureus; pullulanase proteins of Klebsiella pneumoniae and Klebsiella aerogenese; E. coli lipoproteins 1pp-28, Pal, Rp1A, Rp1B, OsmB, NIpB, and Orl17; chitobiase protein of V. harseyi; the β-1,4-endoglucanase protein of Pseudomonas solanacearum, the Pal and Pcp proteins of H. influenzae; the OprI protein of P. aeruginosa; the MalX and AmiA proteins of S. pneumoniae; the 34 kda antigen and TpmA protein of Treponema pallidum; the P37 protein of Mycoplasma hyorhinis; the neutral protease of Bacillus amyloliquefaciens; the 17 kda antigen of Rickettsia rickettsii; the malE maltose binding protein; the rbsB ribose binding protein; phoA alkaline phosphatase; and the OmpA secretion signal (see, e.g., Tanji et al. (1991) J Bacteriol. 173(6):1997-2005). Secretion signal sequences suitable for use in yeast are known in the art, and can be used. See, e.g., U.S. Pat. No. 5,712,113. The rbsB, malE, and phoA secretion signals are discussed in, e.g., Collier (1994) J. Bacteriol. 176:3013.

In some embodiments, a nucleotide sequence encoding subject fusion protein is modified to reflect the codon preference for the particular host cell. For example, the nucleotide sequence will in some embodiments be modified for yeast codon preference. See, e.g., Bennetzen and Hall (1982) J. Biol. Chem. 257(6): 3026-3031. As another non-limiting example, the nucleotide sequence will in other embodiments be modified for E. coli codon preference. See, e.g., Gouy and Gautier (1982) Nucleic Acids Res. 10(22):7055-7074; Eyre-Walker (1996) Mol. Biol. Evol. 13(6):864-872. See also Nakamura et al. (2000) Nucleic Acids Res. 28(1):292.

### Constructs

The present invention further provides recombinant vectors (“constructs”) comprising a subject nucleic acid. In some embodiments, a subject recombinant vector provides for amplification of a subject nucleic acid. In some embodiments, a subject recombinant vector provides for production of an encoded subject fusion protein in a eukaryotic cell, in a prokaryotic cell, or in a cell-free transcription/translation system. Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli, yeast, and plant cells).

Certain types of vectors allow a subject nucleic acid to be amplified. Other types of vectors are necessary for efficient introduction of subject nucleic acid into cells and their stable expression once introduced. Any vector capable of accepting a subject nucleic acid is contemplated as a suitable recombinant vector for the purposes of the invention. The vector may be any circular or linear length of DNA that either integrates into the host genome or is maintained in episomal form. Vectors may require additional manipulation or particular conditions to be efficiently incorporated into a host cell (e.g., an expression plasmid), or can be part of a self-integrating, cell specific system (e.g., a recombinant virus). The vector is in some embodiments functional in a prokaryotic cell, where such vectors function to propagate the recombinant vector and/or provide for expression of a subject nucleic acid and production of a subject fusion protein by the cell. The vector is in some embodiments functional in a eukaryotic cell, where the vector will in many embodiments be an expression vector.

Numerous suitable expression vectors are known to those of skill in the art, and many are commercially available. The following vectors are provided by way of example; for bacterial host cells: pBluescript (Stratagene, San Diego, Calif.), pQE vectors (Qiagen), pBluescript plasmids, pNH vectors, lambda-ZAP vectors (Stratagene); pTrc (Amann et al., Gene, 69:301-315 (1988)); pTrc99a, pKK223-3, pDR540, and pRIT2T (Pharmacia); for eukaryotic host cells: pXT1, pSG5 (Stratagene), pSVK3, pBPV, pMSG, and pSVLSV40 (Pharmacia). However, any other plasmid or other vector may be used so long as it is compatible with the host cell. In particular embodiments, the plasmid vector pSP19g10L is used for expression in a prokaryotic host cell. In other particular embodiments, the plasmid vector pCWori is used for expression in a prokaryotic host cell. See, e.g., Barnes ((1996) Methods Enzymol. 272:1-14) for a description of pSP19g10L and pCWori.

In many embodiments, a subject nucleic acid comprises a nucleotide sequence encoding subject fusion protein, where the subject fusion protein-encoding nucleotide sequence is operably linked to one or more transcriptional and/or translational control elements. Suitable control elements include promoters, e.g., constitutive promoters, inducible promoters, etc.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol., 1991: 173(1): 86-93; Alpuche-Aranda et al., PNAS, 1992; 89(21): 10079-83), a nirB promoter (Harborne et al. (1992) Mol. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., W096/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367-378); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035-7056); and the like.

Non-limiting examples of suitable eukaryotic promoters include CMV immediate early,

HSV thymidine kinase, early and late SV40, LTRs from retrovirus, and mouse metallothionein-I. In some embodiments, e.g., for expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHO5 promoter, a CUP1 promoter, a GAL7 promoter, a MET25 promoter, a MET3 promoter, and the like. Selection of the appropriate vector and promoter is well within the level of ordinary skill in the art. The expression vector may also contain a ribosome binding site for translation initiation and a transcription terminator. The expression vector may also include appropriate sequences for amplifying expression.

A subject recombinant vector will in many embodiments contain one or more selectable marker genes to provide a phenotypic trait for selection of transformed host cells. Suitable selectable markers include, but are not limited to, dihydrofolate reductase, neomycin resistance for eukaryotic cell culture; and tetracycline or ampicillin resistance in prokaryotic host cells such as E. coli.

Generally, recombinant expression vectors will include origins of replication and selectable markers permitting transformation of the host cell, e.g., the ampicillin resistance gene of E. coli, the S. cerevisiae TRP1 gene, etc.; and a promoter derived from a highly-expressed gene to direct transcription of the coding sequence. Such promoters can be derived from operons encoding glycolytic enzymes such as 3-phosphoglycerate kinase (PGK), α-factor, acid phosphatase, or heat shock proteins, among others.

In many embodiments, a nucleotide sequence encoding subject fusion protein is operably linked to an inducible promoter. Inducible promoters are well known in the art. Suitable inducible promoters include, but are not limited to, the pL of bacteriophage λ; Plac; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., P_{BAD} (see, e.g., Guzman et al. (1995) J. Bacteriol. 177:4121-4130); a xylose-inducible promoter, e.g., Pxyl (see, e.g., Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; a heat-inducible promoter, e.g., heat inducible lambda P_{L} promoter, a promoter controlled by a heat-sensitive repressor (e.g., CI857-repressed lambda-based expression vectors; see, e.g., Hoffmann et al. (1999) FEMS Microbiol Lett. 177(2):327-34); and the like.

In yeast, a number of vectors containing constitutive or inducible promoters may be used. For a review see, Current Protocols in Molecular Biology, Vol. 2, 1988, Ed. Ausubel, et al., Greene Publish. Assoc. & Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (Cloning in Yeast, Ch. 3, R. Rothstein In: DNA Cloning Vol. 11, A Practical Approach, Ed. D M Glover, 1986, IRL Press, Wash., D.C.). Alternatively, vectors may be used which promote integration of foreign DNA sequences into the yeast chromosome.

### Compositions

The present invention further provides compositions comprising a subject nucleic acid.

The present invention further provides compositions comprising a subject recombinant vector. Compositions comprising a subject nucleic acid or a subject expression vector will in many embodiments include one or more of: a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; and the like. In some embodiments, a subject nucleic acid or a subject recombinant vector is lyophilized.

### Host Cells

The present invention provides genetically modified host cells, e.g., host cells that have been genetically modified with a subject nucleic acid or a subject recombinant vector. In many embodiments, a subject genetically modified host cell is an isolated in vitro host cell. In other embodiments, a subject genetically modified host cell is an in vivo host cell. In other embodiments, a subject genetically modified host cell is part of a multicellular organism.

Host cells are in many embodiments unicellular organisms, or are grown in in vitro culture as single cells. In some embodiments, the host cell is a eukaryotic cell. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like.

In some embodiments, the host cell is a mammalian cell. Suitable mammalian cells include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In other embodiments, the host cell is a plant cell. Plant cells include cells of monocotyledons (“monocots”) and dicotyledons (“dicots”). Guidance with respect to plant tissue culture may be found in, for example: Plant Cell and Tissue Culture, 1994, Vasil and Thorpe Eds., Kluwer Academic Publishers; and in: Plant Cell Culture Protocols (Methods in Molecular Biology 111), 1999, Hall Eds, Humana Press.

In other embodiments, the host cell is a prokaryotic cell. Suitable prokaryotic cells include, but are not limited to, any of a variety of laboratory strains of Escherichia coli, Lactobacillus sp., Salmonella sp., Shigella sp., and the like. See, e.g., Carrier et al. (1992) J. Immunol. 148:1176-1181; U.S. Pat. No. 6,447,784; and Sizemore et al. (1995) Science 270:299-302. Examples of Salmonella strains which can be employed in the present invention include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some embodiments, the host cell is Escherichia coli.

To generate a subject genetically modified host cell, a subject nucleic acid comprising nucleotide sequences encoding a subject fusion protein is introduced stably or transiently into a parent host cell, using established techniques, including, but not limited to, electroporation, calcium phosphate precipitation, DEAE-dextran mediated transfection, liposome-mediated transfection, and the like. For stable transformation, a nucleic acid will generally further include a selectable marker, e.g., any of several well-known selectable markers such as neomycin resistance, ampicillin resistance, tetracycline resistance, chloramphenicol resistance, kanamycin resistance, and the like.

A subject genetically modified host cell is in many embodiments cultured in vitro in a suitable medium and at a suitable temperature. The temperature at which the cells are cultured is generally from about 18° C. to about 40° C., e.g., from about 18° C. to about 20° C., from about 20° C. to about 25° C., from about 25° C. to about 30° C., from about 30° C. to about 35° C., or from about 35° C. to about 40° C. (e.g., at about 37° C.).

In some embodiments, a subject genetically modified host cell is cultured in a suitable medium (e.g., Luria-Bertoni broth, optionally supplemented with one or more additional agents, such as an inducer (e.g., where the subject fusion protein-encoding nucleotide sequence is under the control of an inducible promoter), etc.). After a suitable time, the subject fusion protein is isolated from the cell lysate and/or cell culture medium.

### Silica Matrices

Functionalized molecules comprising an autosilification moiety become immobilized in a silica matrix upon reaction with a silicic acid in an appropriate buffer. The present invention further provides a silica matrix comprising a subject functionalized molecule. A subject silica matrix can be of any of a variety of forms, including, e.g., spheres, sheets, fibrils, etc. The form of the matrix will depend on various factors, e.g., the nature of the functionalized molecule immobilized therein, the temperature at which the reaction of the functionalized molecule with silicic acid is carried out, the concentration of the buffer in which the reaction of the functionalized molecule with silicic acid is carried out, etc.

In some embodiments, a subject silica matrix is spherical, and the individual spheres have an average diameter of from about 10 nm to about 1000 nm, e.g., from about 10 nm to about 20 nm, from about 20 nm to about 30 nm, from about 30 nm to about 40 nm, from about 40 nm to about 50 nm, from about 50 nm to about 100 nm, from about 100 nm to about 150 nm, from about 150 nm to about 200 nm, from about 200 nm to about 250 nm, from about 250 nm to about 300 nm, from about 300 nm to about 400 nm, from about 400 nm to about 500 nm, from about 500 nm to about 600 nm, from about 600 nm to about 700 nm, from about 700 nm to about 800 nm, from about 800 nm to about 900 nm, or from about 900 nm to about 1000 nm.

In some embodiments, a subject silica matrix is in the form of elongated fibers, and the fibers have an average diameter of from about 10 nm to about 1000 nm, e.g., from about 10 nm to about 20 nm, from about 20 nm to about 30 nm, from about 30 nm to about 40 nm, from about 40 nm to about 50 nm, from about 50 nm to about 100 nm, from about 100 nm to about 150 nm, from about 150 nm to about 200 nm, from about 200 nm to about 250 nm, from about 250 nm to about 300 nm, from about 300 nm to about 400 nm, from about 400 nm to about 500 nm, from about 500 nm to about 600 nm, from about 600 nm to about 700 nm, from about 700 nm to about 800 nm, from about 800 nm to about 900 nm, or from about 900 nm to about 1000 nm.

In some embodiments, a subject matrix is in the form of a sheet, e.g., forms a planar surface, where the sheet has a thickness of from about 10 nm to about 1000 nm, e.g., from about 10 nm to about 20 nm, from about 20 nm to about 30 nm, from about 30 nm to about 40 nm, from about 40 nm to about 50 nm, from about 50 nm to about 100 nm, from about 100 nm to about 150 nm, from about 150 nm to about 200 nm, from about 200 nm to about 250 nm, from about 250 nm to about 300 nm, from about 300 nm to about 400 nm, from about 400 nm to about 500 nm, from about 500 nm to about 600 nm, from about 600 nm to about 700 nm, from about 700 nm to about 800 nm, from about 800 nm to about 900 nm, or from about 900 nm to about 1000 nm.

In some embodiments, a subject matrix is in the form of an arch (e.g., a parabolic arch structure). An arch structure can be formed by repeated addition (e.g., serial addition) of silica spheres.

In some embodiments, a subject matrix is in the form of a hexagon, where a side of the hexagon is from about 10 nm to about 10 pm, e.g., from about 10 nm to about 1000 nm, e.g., from about 10 nm to about 20 nm, from about 20 nm to about 30 nm, from about 30 nm to about 40 nm, from about 40 nm to about 50 nm, from about 50 nm to about 100 nm, from about 100 nm to about 150 nm, from about 150 nm to about 200 nm, from about 200 nm to about 250 nm, from about 250 nm to about 300 nm, from about 300 nm to about 400 nm, from about 400 nm to about 500 nm, from about 500 nm to about 600 nm, from about 600 nm to about 700 nm, from about 700 nm to about 800 nm, from about 800 nm to about 900 nm, from about 900 nm to about 1 μm, from about 1 μm to about 5 μm, or from about 5 μm to about 10 μm.

In some embodiments, a subject matrix has an ellipsoid form, a toroidal form, an ovoid form, a cube form, a tubular form, a cylindrical form, a tear shape, and the like.

In some embodiments, a subject matrix has a repeat structure, e.g., comprises a plurality of subunits, where the subunits may be triangular, square, pentagonal, hexagonal, octagonal, circular, etc. In some embodiments, the repeat structure includes periodic gaps between the subunits. In some embodiments, a subject matrix is ladder-shaped, with periodic gaps between the “rungs” (which “rungs” can be cylindrical); e.g., the average size of the gap ranges from about 10 nm to about 1000 nm.

Functionalized molecules present in a subject silica matrix can be present in the matrix in random order, or are present in an ordered fashion.

In some embodiments, functionalized molecules are present in a subject silica matrix in an order. For example, in some embodiments, the functionalized molecules are arranged into an ordered array in a planar (sheet) silica matrix. In other embodiments, individual silica spheres are ordered in an array. The array can contain a number of different biomolecules. Such an array can be used to detect an analyte in a sample, to provide for sequential enzymatic modification of a substrate, etc.

The array may have a plurality of addresses. For example, a subject silica matrix array can have a density of at least 10, 10², 10³, or 10⁴, or more addresses per cm². Each address can contain 1 mg, 1 μg, 1 ng, 100 pg, 10 pg, 0.1 pg, or less of a functionalized molecule, or any amount in between. Alternatively, each address can contain 10², 10⁴, 10⁶, 10⁸, or more functionalized molecules, or any amount in between. Different addresses (e.g., different demarcated regions) may have the same or different amounts of functionalized molecules. Each address can be directly adjacent to at least one another address. Alternatively, the addresses can be separated from each other, e.g., by a ridge or by an etch. The addresses can be distributed on the silica matrix in one dimension, e.g., a linear array; or in two dimensions, e.g., a rectangular array.

In some embodiments, a subject silica matrix is in the form of spheres that are provided in a column.

A subject silica matrix can be further modified with one or more magnetic particles. In some embodiments, a subject silica matrix is further modified to comprise one or more particles that are detectable via magnetic resonance imaging, e.g., ferrites of general composition MeOₓFe₂O₃ wherein Me is a bivalent metal such as Co, Mn or Fe; γ-Fe₂O₃; the pure metals Co, Fe, Ni; and metal compounds such as carbides and nitrides.

In some embodiments, a subject silica matrix will include a moiety that generates a detectable signal. For example, in some embodiments a subject silica matrix will comprise a functionalized molecule that itself generates a detectable signal, e.g., a fluorescent protein, a detectably labeled antibody, etc.

In some embodiments, a subject silica matrix will comprise a single (e.g., only one) functionalized molecule. In other embodiments, a subject silica matrix will include two, three, four, five, six, seven, eight, nine, ten, or more, different functionalized molecules.

### Methods of Making a Silica Matrix

To prepare a subject silica matrix, a functionalized molecule is reacted with silicic acid in the presence of a suitable buffer. The present invention thus provides methods of making a subject silica matrix. Silicic acids suitable for use in a subject method of making a silica matrix generally have the formula [SiOₓ(OH)₄₋₂ₓ]ₙ. Suitable silicic acids include, but are not limited to, metasilicic acid (H₂SiO₃), orthosilicic acid (H₄SiO₄), pyrosilicic acid (H₆Si₂O₇), hydrolyzed tetramethyl orthosilicate, and the like.

The reaction is carried out in a suitable buffer. Suitable buffers include, but are not limited to, phosphate buffers and sulfate buffers. Suitable phosphate buffers include, but are not limited to, potassium phosphate buffers, sodium phosphate buffers, and the like. Suitable concentration ranges of the buffer include from about 10 mM to about 1 M, e.g., from about 10 mM to about 20 mM, from about 20 mM to about 50 mM, from about 50 mM to about 100 mM, from about 100 mM to about 250 mM, from about 250 mM to about 500 mM, from about 500 mM to about 750 mM, or from about 750 mM to about 1 M.

For example, in some embodiments, the reaction is carried out in a phosphate buffer having a concentration of from about 1M KH₂PO₄ and 1N NaOH, pH 8, to about 12.5 mM KH₂PO₄ and 12.5 mN NaOH, pH 8.

The reaction is carried out at a temperature of from about 0° C. to about 98° C., e.g., from about 0° C. to about 5° C., from about 5° C. to about 10° C., from about 10° C. to about 15° C., from about 15° C. to about 22° C., from about 22° C. to about 25° C., from about 25° C. to about 30° C., from about 30° C. to about 40° C., from about 40 ° C. to about 50° C., from about 50° C. to about 60° C., from about 60° C. to about 70° C., from about 70° C. to about 80° C., from about 60° C. to about 90° C., or from about 90° C. to about 98° C.

### Utility

A subject silica matrix finds use in a wide variety of applications, which are provided by the present invention. Exemplary applications include research applications; bioreactor applications; analyte detection applications; diagnostic applications; screening applications; purification methods; and therapeutic applications.

### Bioreactor Applications

In some embodiments, a subject silica matrix functions as a bioreactor, e.g., the silica matrix comprises one or more functionalized enzymes (enzymes comprising a covalently linked autosilification moiety) that catalyze the conversion of a substrate or an intermediate to yield a product of interest. In these embodiments, a subject matrix is also referred to as a “catalytic matrix.”

For example, in some embodiments, a subject matrix comprises one or more biosynthetic pathway enzymes. In some embodiments, as described above, a subject silica matrix comprises one, two, three, four, five, six, seven, eight, nine, ten, or more, enzymes in a synthetic pathway. Suitable biosynthetic pathways include pathways comprising enzymes that catalyze the synthesis of a compound of interest, where compounds of interest include, but are not limited to, isoprenoids, terpenoids, tetrapyrroles, polyketides, macrolides, vitamins, amino acids, fatty acids, proteins, nucleic acids, carbohydrates, biopolymers, antimicrobial agents, and anticancer agents.

In some embodiments, a subject silica matrix comprising one or more biosynthetic pathway enzymes is contacted with a substrate or starting material, and the enzymes present in the silica matrix convert the substrate to a product. In some embodiments, the product is then collected. The product can be subjected to one or more of concentration, further purification steps (e.g., to remove intermediates), chemical modification, enzymatic modification, etc. A subject catalytic matrix provides certain advantages over synthesizing a product of interest in a living cell. For example, use of a subject catalytic matrix avoids possible inhibition of an enzyme in a biosynthetic pathway that may occur in a living cell due to accumulation of toxic levels of an intermediate in the pathway. Use of a subject catalytic matrix also provides products that require little, if any, further purification steps, as no cellular components (other than the biosynthetic pathway enzymes) are present. A further advantage is that the reaction need not be carried out under physiological conditions.

In some embodiments, an enzymatic reaction, or series of enzymatic reactions, is carried out using a subject catalytic matrix, where the reactions are carried out under physiological conditions, e.g., a temperature in the range of from about 25° C. to about 40° C., a pH in the range of from about 6.5 to about 8.0, and in an aqueous solution, e.g., a buffered aqueous solution. Suitable aqueous solutions can include one or more of a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.

In other embodiments, an enzymatic reaction, or series of enzymatic reactions, is carried out using a subject catalytic matrix, where the reactions are carried out under non-physiological conditions. Non-physiological conditions include one or more of: 1) a temperature lower than about 25° C.; 2) a temperature higher than about 40° C. (e.g., from about 40° C. to about 50° C., from about 50° C. to about 60° C., from about 60° C. to about 75° C., or from about 75° C. to about 100° C., or higher); 3) a non-aqueous solution, e.g., an organic solvent; 4) inclusion of detergents (e.g., non-ionic detergent such as Tween-20, and the like; ionic detergents); 5) inclusion of a solubilization agent; 6) a higher than physiological salt concentration (e.g., a salt concentration of from about 50 mM to about 100 mM, from about 100 mM to about 150 mM, from about 150 mM to about 200 mM, from about 200 mM to about 300 mM, from about 300 mM to about 500 mM, from about 500 mM to about 1 M, or higher). In some embodiments, an enzymatic reaction, or series of enzymatic reactions, is carried out using a subject catalytic matrix, where the reactions are carried out in a non-aqueous solution, e.g., in an organic solvent. Organic solvents include, but are not limited to, toluene, dodecane,benzene, carbon tetrachloride, hexane, methanol, ethanol, n-propanol, isopropanol, and the like.

In some embodiments, an enzymatic reaction, or series of enzymatic reactions, is carried out using a subject catalytic matrix, where the reactions are carried out at a temperature that is in the range of the temperature optimum of the enzyme(s) in the matrix. In some embodiments, an enzymatic reaction, or series of enzymatic reactions, is carried out using a subject catalytic matrix, where the reactions are carried out at a pH that is in the range of the pH optimum of the enzyme(s) in the matrix.

In some embodiments, a subject catalytic matrix comprises one, two, three, four, five, six, seven, eight, nine, ten, or more, enzymes in a biosynthetic pathway. For example, suitable enzymes include enzymes that catalyze the synthesis of isoprenoid compounds via a mevalonate pathway. See, e.g., U.S. Patent Application Nos. 2003/0148479 and 2006/0079476. Enzymes that catalyze the synthesis of isopentenyl pyrophosphate, the universal intermediate in the synthesis of a wide variety of isoprenoid compounds, include acetoacetyl-CoA thiolase, hydroxymethylglutaryl-CoA (HMG-CoA) synthase (HMGS), HMG-CoA reductase (HMGR), mevalonate kinase (MK), phosphomevalonate kinase (PMK), and mevalonate pyrophosphate decarboxylase (MPD). A subject silica matrix can include all six of these enzymes; and the matrix can be contacted with acetyl-CoA, which is converted by the enzymes to isopentenyl pyrophosphate (IPP). In another embodiment, the silica matrix includes the MK, PMK, and MPD enzymes, and does not include the acetoacetyl-CoA thiolase, the HMGS, or the HMGR; and the matrix is contacted with mevalonate, which is converted by the MK, PMK, and MPD to IPP. The matrix can further include an IPP isomerase, which converts IPP to dimethylallyl diphosphate (DMAPP). The matrix can further include one or more of a prenyl transferase (e.g., geranyl pyrophosphate synthase; farnesyl pyrophosphate synthase; geranylgeranyl pyrophosphate synthase; hexadecylpyrophosphate synthase; octaprenyl pyrophosphate synthase; nonaprenyl pyrophosphate synthase; and decaprenyl pyrophosphate synthase); and a terpene synthase (e.g., amorphadiene synthase; (−)-germacrene D synthase; E,E-alpha-farnesene synthase; 1,8-cineole synthase; pinene synthase; (E)-β-ocimene synthase; (−)-camphene synthase; (−)-4S-limonene synthase; delta-selinene synthase; E-α-bisabolene synthase; gamma-humulene synthase; δ-selinene synthase; and the like).

As another non-limiting example, a subject catalytic matrix includes enzymes that catalyze the synthesis of terephthalic acid. See, e.g., U.S. Pat. No. 6,461,840 for enzymes that catalyze the synthesis of terephthalic acid. As another non-limiting example, a subject catalytic matrix includes enzymes that catalyze the synthesis of everninomycin; see, e.g., U.S. Pat. No. 6,861,513 for enzymes that catalyze the synthesis of everninomycin. As another non-limiting example, a subject catalytic matrix includes enzymes that catalyze the synthesis of an L-amino acid; see, e.g., U.S. Pat. No. 7,037,690. As another non-limiting example, a subject catalytic matrix includes enzymes that catalyze the synthesis of a polyketide; see, e.g., U.S. Pat. Nos. 7,078,233, 7,067,290 and 7,022,825 for enzymes that catalyze the synthesis of polyketides.

In some embodiments, a subject catalytic matrix includes enzymes that catalyze synthesis of one or more alkaloids.

In some embodiments, a subject catalytic matrix includes one or more enzymes that provide for detoxification of a toxic compound. In some of these embodiments, the matrix is present in a filter (e.g., a circular, rectangular, or square filter) or a column, where a liquid sample comprising a toxic compound is applied to a first side of the filter, or to the top of the column; the enzyme(s) present in the matrix carry out one or more enzymatic reactions that detoxifies the toxic compound (e.g., removes one or more moieties from the toxin or otherwise alters the structure of the toxin such that the toxicity of the toxic compound is reduced or eliminated). Liquid comprising the detoxified compound is collected. In these embodiments, a subject matrix is useful for detoxifying a liquid sample. Thus, in some embodiments, the present invention provides methods for reducing or eliminating the toxicity of a toxic compound, the method generally involving contacting a liquid sample comprising one or more toxic compounds to a subject matrix, where the matrix comprises one or more enzymes that detoxify a toxic compound, thereby generating a compound with reduced toxicity.

In some embodiments, a subject catalytic matrix is disposed within a column or other support. In these embodiments, a first end of the column includes an opening or a port for introduction of a substrate. A second end includes a mesh or other porous support for retaining the catalytic matrix. The second end includes, distal to the porous support, an opening for release of product of enzymatic reaction(s) on the substrate. For example, a subject catalytic matrix can be disposed within a column, in a liquid (e.g., an aqueous solvent, an organic solvent, etc.). Substrate is introduced into the first end of the column. Product is collected in fractions at the second end of the column. In some embodiments, the column is sized as an industrial-scale bioreactor, e.g., for use in large-scale (e.g., milligram to gram quantities; e.g., from about 1 mg to about 1 gram or more than 1 gram) production of a product. In other embodiments, the column is sized for use in a laboratory setting, e.g., for small scale production (e.g., picogram to nanograms, nanogram to microgram, or microgram to milligram; e.g., from about 1 pg to about 1 ng, from about 1 ng to about 1 μg, or from about 1 μg to about 1 mg) of a product.

### Analyte Detection Applications

In some embodiments, a subject silica matrix functions as an analyte detector. In these embodiments, a subject silica matrix is also referred to as a “silica analyte detector matrix.” A subject silica analyte detector matrix is useful for detecting a wide variety of analytes in a sample, for a wide variety of applications.

In some embodiments, a subject silica analyte detector matrix comprises a functionalized molecule that provides for binding to an analyte, where such a functionalized molecule is referred to as a “capture agent” or an “analyte binding moiety.” In some embodiments, the capture agent is an antibody.

In carrying out a subject analyte detection method, a subject silica analyte detector matrix is contacted with a sample; and binding of molecule(s) in the sample to the silica analyte detector matrix is detected.

In some embodiments, a first antibody specific for a first analyte is present in a first silica matrix; and a second antibody specific for a second analyte is present in a second silica matrix, and the first and second antibodies are detectably labeled with labels that are distinguishable from one another.

In general, a test sample is compared to a negative control sample and a positive control sample, where a negative control sample does not contain the analyte in any detectable amount, and a positive control sample is a sample known to contain a detectable amount of the analyte being detected. A plurality of positive control samples may be used, where each contains the analyte at a different concentration.

### Diagnostic Applications

In some embodiments, a subject silica matrix is useful in diagnostic applications.

Diagnostic applications include detection of the presence of a cancerous cell in a patient or in a biological sample obtained from a patient; detection of a metabolite in a patient or in a biological sample obtained from a patient; detection of an abnormal level of a marker in a patient or in a biological sample obtained from a patient; and the like.

As one non-limiting example, a subject silica matrix that includes an antibody that binds specifically to a marker present on the surface of a cancer cell (e.g. a cancer cell-specific marker) is used to detect the presence and/or location of a cancer cell in a patient or in a biological sample obtained from the patient. In some embodiments, the silica matrix includes one or more moieties that provide for detection of binding of the silica matrix to a cell.

In some embodiments, two or more samples are tested. The different samples may consist of an “experimental” (or “test”) sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell type. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells.

### Screening Methods

In some embodiments, a subject silica matrix is useful in a screening method, e.g., a method of identifying an agent that modulates the function of a biomolecule. The methods generally involve contacting a subject silica matrix with a test agent, and determining the effect, if any, of the test agent on the activity of the functionalized molecule in the matrix. For example, a subject silica matrix that comprises an enzyme can be used to test the effect, if any, of a test agent on the activity of the enzyme. As another example, a subject silica matrix that comprises a ligand-gated ion channel can be used to test the effect, if any, of a test agent on the function of the ion channel.

Assays of the invention include controls, where suitable controls include a sample (e.g., a sample comprising a subject silica matrix in the absence of the test agent). Generally a plurality of assay mixtures is run in parallel with different agent concentrations to obtain a differential response to the various concentrations. Typically, one of these concentrations serves as a negative control, i.e. at zero concentration or below the level of detection.

The terms “candidate agent,” “test agent,” “agent,” “substance,” and “compound” are used interchangeably herein. Candidate agents encompass numerous chemical classes, in some embodiments synthetic, semi-synthetic, or naturally occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.) and can also be used. Alternatively, libraries of natural compounds in the form of bacterial, fungal, plant and animal extracts are available from Pan Labs (Bothell, Wash.) or are readily producible.

Candidate agents are in some embodiments small organic or inorganic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

A variety of other reagents may be included in the screening assay. These include reagents like salts, neutral proteins, e.g. albumin, detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used. The components of the assay mixture are added in any order that provides for the requisite binding or other activity. Incubations are performed at any suitable temperature, typically between 4° C. and 40° C. Incubation periods are selected for optimum activity, but may also be optimized to facilitate rapid high-throughput screening. Typically between 0.1 hour and 1 hour will be sufficient.

### Purification Methods

In some embodiments, a subject silica matrix is useful for isolated and/or purifying one or more molecules from a sample. For example, where a subject silica matrix comprises a functionalized molecule that is a first member of a specific binding pair, the second member of the specific binding pair can be isolated from a sample by virtue of selective binding of the second member to the first member.

For example, a subject silica matrix that comprises a first member of a specific binding pair is contacted with a sample that comprises a second member of the specific binding pair under conditions that permit binding of the second member to the first member, generating a second member-bound silica matrix. The second member-bound silica matrix can be then removed from the sample by centrifugation. Alternatively the silica matrix is immobilized, and, following binding of the second member to the immobilized silica matrix, the remainder of the sample is simply washed away from the second member-bound silica matrix. Immobilization of the silica matrix is accomplished in a number of ways. For example, the silica matrix can be in the form of spheres that are packed in a cylinder (a column) comprising a filter of mesh at one end that retains the spheres.

### Therapeutic Applications

In some embodiments, a subject silica matrix is useful in various therapeutic applications. For example, where a subject silica matrix comprises a drug, the silica matrix is administered to an individual in need of the drug (therapeutic agent).

In some embodiments, the silica matrix comprises: a) a targeting moiety that provides for targeting to a particular cell (e.g., an antibody specific for a cancer cell); and b) a therapeutic agent (drug) that acts on the targeted cell. In other embodiments, the silica matrix comprises: a) a therapeutic agent; and b) a detection moiety, e.g., a moiety that is detectable by MRI. In other embodiments, the silica matrix comprises: a) a therapeutic agent; and b) a moiety that provides for crossing the blood-brain barrier.

### Research Applications

In some embodiments, a subject silica matrix is useful in various research applications.

As one non-limiting example, a subject silica matrix comprises multiple enzymes in a biosynthetic pathway, and the silica matrix is used to study regulation of the pathway. In other embodiments, a subject matrix is a mesoporous silica matrix that functions as a semipermeable membrane for the study of various biological activities such as signal transduction.

### Devices and Kits

The present invention further provides devices and kits comprising a subject silica matrix. A subject device, or a subject kit, is useful for carrying out a subject method.

### Devices

The present invention provides a device comprising a subject silica matrix. The device can have any of a variety of forms, including, but not limited to, a column, a tube, a plate, a chip, etc., where the form of the device will depend, in part, on the use for which the silica matrix is intended. The device can be a spin column, a “microfuge” tube, or other tube or column that comprises a subject silica matrix.

In some embodiments, a subject device is a column or other support. In these embodiments, a first end of the column includes an opening or a port for introduction of a substrate for an enzyme(s) present in the matrix, or a sample comprising an analyte to be purified on the matrix. A second end includes a mesh or other porous support for retaining the matrix. The second end includes, distal to the porous support, an opening for release of product of enzymatic reaction(s) on the substrate, or for release of unbound materials, or for release of eluted analyte.

For example, a subject catalytic matrix can be disposed within a column, in a liquid (e.g., an aqueous solvent, an organic solvent, etc.). Substrate is introduced into the first end of the column. Product is collected in fractions at the second end of the column. As another example, a sample comprising a mixture of substances, including an analyte(s) to be purified using a subject matrix, is introduced into the first end of the column. Unbound material is washed through, after which bound analyte is eluted from the column.

In some embodiments, the column is sized as an industrial-scale bioreactor, e.g., for use in large-scale (e.g., milligram to gram quantities; e.g., from about 1 mg to about 1 gram or more than 1 gram) production of a product. In other embodiments, the column is sized for use in a laboratory setting, e.g., for small scale production (e.g., picogram to nanograms, nanogram to microgram, or microgram to milligram; e.g., from about 1 pg to about 1 ng, from about 1 ng to about 1 μg, or from about 1 μg to about 1 mg) of a product.

### Kits

The present invention further provides kits for carrying out a subject method. In some embodiments, a subject kit will include at least a subject functionalized molecule, or a subject matrix. In some embodiments, a subject kit will include a subject device.

Where a subject kit provides components for carrying out an enzymatic reaction, or a series of enzymatic reaction, the kit will include a subject catalytic matrix. Such a kit can further include one or more of a substrate, wash buffers, aqueous or non-aqueous solvents, standards, and positive and negative controls. The kit can further include a column, for packing with the catalytic matrix; or a column pre-packed with the catalytic matrix.

Where a subject kit provides components for carrying out isolation and/or purification of an analyte from a sample, the kit will include a subject matrix that comprises a covalently attached member of a specific binding pair, as described above. Such a kit can further include one or more of a binding buffer, a wash buffer, an elution buffer, standards, and the like.

In addition to the above components, the subject kits will in some embodiments further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, compact disk (CD), digital versatile disk, etc., on which the information has been recorded. Yet another means that may be present is a website address which may be used via the internet to access the information at a removed site. Any convenient means may be present in the kits.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Silaffin-EAK Fusions

### Materials and Methods

Matrix Formation: Protein stock solutions were made by diluting each peptide to 100 mg/mL. Tetramethyl-orthosilicate (TMOS) was diluted to 1M in 1 mM HCl and allowed to hydrolyze for 15 minutes at room temperature. A 1× phosphate buffer of 0.1 M KH₂PO₄ and 0.1 N NaOH was also made.

To produce the matrices, the peptide, phosphate buffer, and hydrolyzed TMOS were mixed. A 10 μL reaction mixture consisted of 8 μL, phosphate buffer, 1 μL peptide, and 1 μL hydrolyzed TMOS.

The order of mixing of the components and reaction temperature resulted in various matrix morphologies.

Matrices were analyzed by scanning electron microscopy (SEM) and traditional light microscopy.

The R5 silaffin peptide used has the amino acid sequence: NH₂-SSKKSGSYSGSKGSKRRIL-COOH (SEQ ID NO:1). The EAK₁ peptide used has the sequence: NH₂-AEAEAKAKAEAEAKAK-COOH (SEQ ID NO:39). The EAK₁ peptide displays the properties of a hydrogel. The R5 silaffin peptide was fused to the carboxyl terminus of the EAK₁ peptide, resulting in the R5 silaffin/EAK fusion having the sequence:

NH₂-AEAEAKAKAEAEAKAKSSKKSGSYSGSKGSKRRIL-COOH (SEQ ID NO:30). This peptide was produced using standard chemical peptide synthetic methods.

A peptide encoded by the plasmid pET30 was also fused to the R5 silaffin peptide. The pET30-encoded peptide has the amino acid sequence: NH₂-MHHHHHHSSGLVPRGSGMKETAAAKFERQHMDSPDLGTDDDDKAMGYLWIRIRAPS TSLRPHSSTTTTTTEIRLLTKPERKLSWLLPPLSNN—COOH (SEQ ID NO:31). A nucleotide sequence encoding the R5-silaffin peptide was inserted in-frame into the nucleotide sequence encoding the pET30 peptide, resulting in an R5 silaffin-pET30 fusion peptide having the amino acid sequence: NH₂-MHHHHHHSSGLVPRGSGMKETAAAKFERQHMDSPDLGTDDDDKAMA SSKKSGSYSGSKGSKRRIL-COOH (SEQ ID NO:32). The R5 silaffin-pET30 fusion peptide was produced in a genetically modified Escherichia coli. E. coli strain BLR(DE3) harboring the pET30-R5 plasmid was grown to an OD₆₀₀ of 0.8, when protein expression was induced via the addition of 1 mM IPTG. After 3 hours of protein expression, the cells were harvested via centrifugation. Cell pellets were resuspended in buffer and lysed by sonication. Insoluble material was removed by centrifugation, and the recombinant R5 peptide was purified using immobilized metal affinity chromatography (IMAC). For the IMAC, protein was loaded onto a column containing immobilized nickel ions and then washed in purification buffer with increasing concentrations of imidazole until a purified sample of recombinant R5 eluted from the column.

### Results

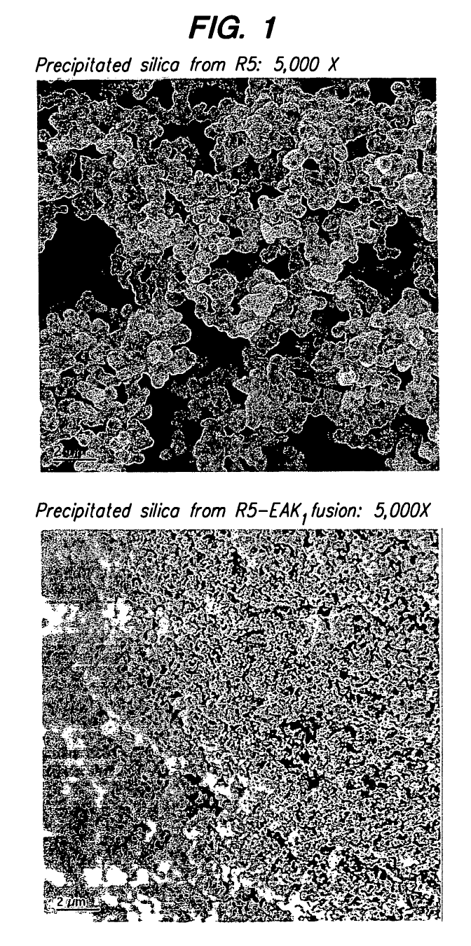

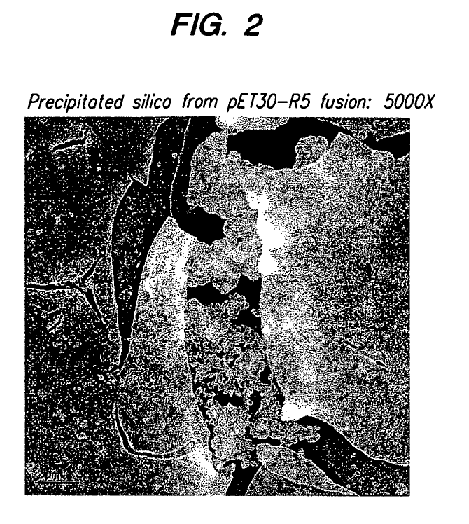

The results are shown in FIGS. 1 and 2. FIG. 1 is a scanning electron micrograph of silica matrices formed using chemically synthesized R5 peptide (top) and R5-EAK₁ fusion peptide (bottom). Silica matrices formed using the R5 peptide forms silica spheres with diameters of approximately 500 nm. Matrices formed using R5-EAK form an interconnected matrix of spheres with much smaller diameters. FIG. 2 is a scanning electron micrograph of the silica matrices formed by recombinantly produced R5 peptide from the pET30-R5 plasmid. Note that the size of the silica spheres is not monodisperse, but that spheres of diameter 500 nm are formed.

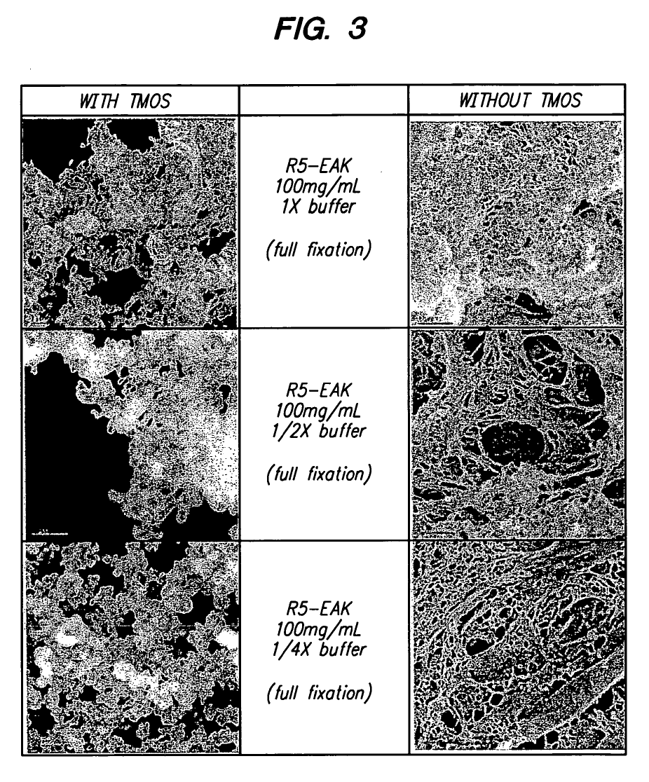

The morphology of the R5 silaffin-EAK₁ silica deposits produced under various processing conditions was analyzed. As shown in FIG. 3, R5-EAK₁ silica deposits formed with and without TMOS and at various concentrations of phosphate buffer display very different morphologies. For samples with TMOS, the formation of both spheres and filaments is evident. Note that as the concentration of the phosphate buffer decreases, the polydispersity of the silica spheres increases. For samples without TMOS, the fibril structure of the EAK domain is evident, but the formation of silica spheres within the domain is not.

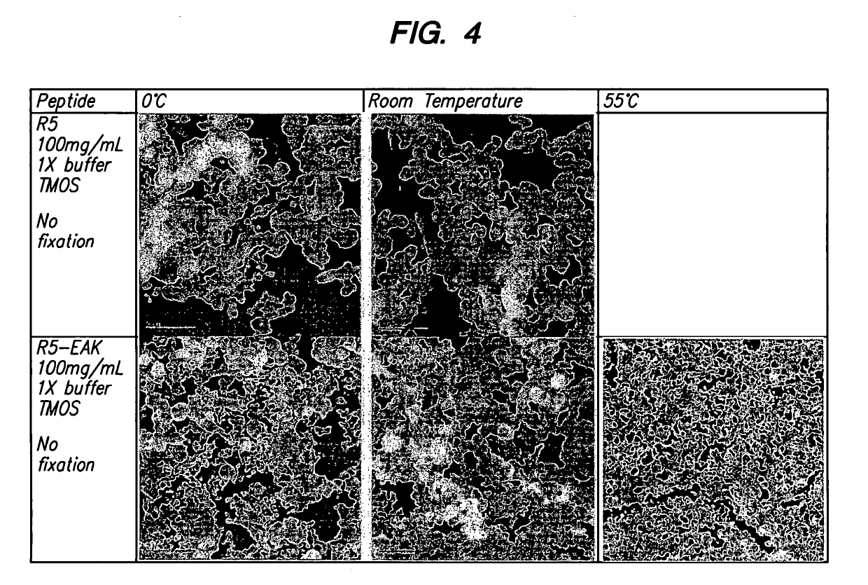

The impact of processing temperature on silica morphology was evaluated. R5-EAK₁ silica deposits were formed at a peptide concentration of 100 mg/ml in 1× buffer and TMOS at 0° C., room temperature (about 22° C.), and at 55° C. The results are shown in FIG. 4. For a given reaction time, the reaction temperature appears to influence the size dispersity of the spheres in the final matrix. For the R5 peptide, lower temperature result in a lower number of fully grown (diameter=500 nm) spheres. The R5-EAK peptide seems to have a greater dispersity in sphere sizes as well, but with a greater degree of densely packed smaller spheres as temperature increases.

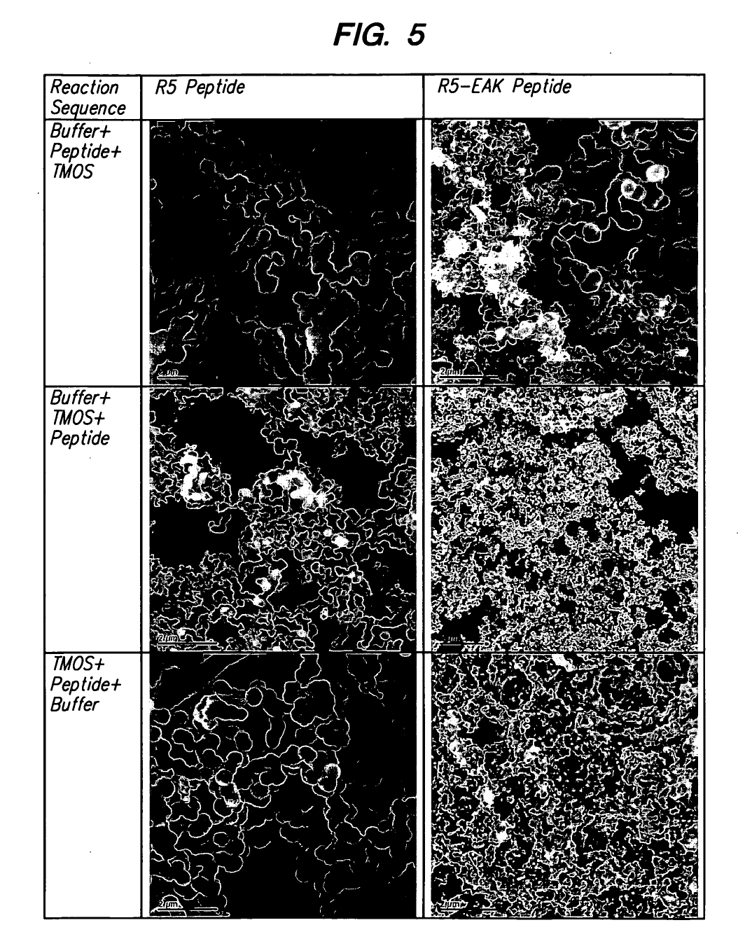

Finally, the impact of reaction sequence on silica morphology was evaluated. R5-EAK₁ silica deposits were formed by mixing the reagents in the following orders: 1) buffer+peptide+TMOS; 2) buffer+TMOS+peptide; and 3) TMOS+peptide+buffer. The results are shown in FIG. 5. Reaction order seems to have little impact on the morphology of matrices formed by R5 peptide, but it does have an impact on the EAK-R5 matrices. For the Buffer+Peptide+TMOS and buffer+TMOS+Peptide case, the morphology consists of a polydisperse mixture of interconnected spheres. For the TMOS+peptide+buffer case, the matrix consists of a network of thick silica filaments with no visible sphere morphology.

### Example 2

### Silaffin-GFP Fusions

Silaffin-GFP fusions were made expressing the two proteins in frame with each other using a pET30 expression vector in E. coli BLR(DE3).

The nucleotide sequence encoding the fusion protein is as follows:

(SEQ ID NO: 33)

atgcaccatcatcatcatcattcttctggtctggtgccacgcggttctgg
tatgaaagaaaccgctgctgctaaattcgaacgccagcacatggacagcc
cagatctgggtaccgacgacgacgacaaggccatggcttcttcctctaaa
aagtctggttcctactctggtagcaaaggctccaaacgtcgcatcctggc
cagtaaaggagaagaacttttcactggagttgtcccaattcttgttgaat
tagatggtgatgttaatgggcacaaattttctgtcagtggagagggtgaa
ggtgatgcaacatacggaaaacttacccttaaatttatttgcactactgg
aaaactacctgttccatggccaacacttgtcactactttctcttatggtg
ttcaatgcttttcccgttatccggatcatatgaaacggcatgactttttc
aagagtgccatgcccgaaggttatgtacaggaacgcactatatctttcaa
agatgacgggaactacaagacgcgtgctgaagtcaagtttgaaggtgata
cccttgttaatcgtatcgagttaaaaggtattgattttaaagaagatgga
aacattctcggacacaaactcgagtacaactataactcacacaatgtata
catcacggcagacaaacaaaagaatggaatcaaagctaacttcaaaattc
gccacaacattgaagatggatccgttcaactagcagaccattatcaacaa
aatactccaattggcgatggccctgtccttttaccagacaaccattacct
gtcgacacaatctgccctttcgaaagatcccaacgaaaagcgtgaccaca
tggtccttcttgagtttgtaactgctgctgggattacacatggcatggat
gagctctacaaataa.

The amino acid sequence of the fusion protein is as follows:

(SEQ ID NO: 34)

M H H H H H H S S G L V P R G S G M K E T A A A K
F E R Q H M D S P D L G T D D D D K A M A S S S K
K S G S Y S G S K G S K R R I L A S K G E E L F T
G V V P I L V E L D G D V N G H K F S V S G E G E
G D A T Y G K L T L K F I C T T G K L P V P W P T
L V T T F S Y G V Q C F S R Y P D H M K R H D F F
K S A M P E G Y V Q E R T I S F K D D G N Y K T R
A E V K F E G D T L V N R I E L K G I D F K E D G
N I L G H K L E Y N Y N S H N V Y I T A D K Q K N
G I K A N F K I R H N I E D G S V Q L A D H Y Q Q
N T P I G D G P V L L P D N H Y L S T Q S A L S K
D P N E K R D H M V L L E F V T A A G I T H G M D
E L Y K.

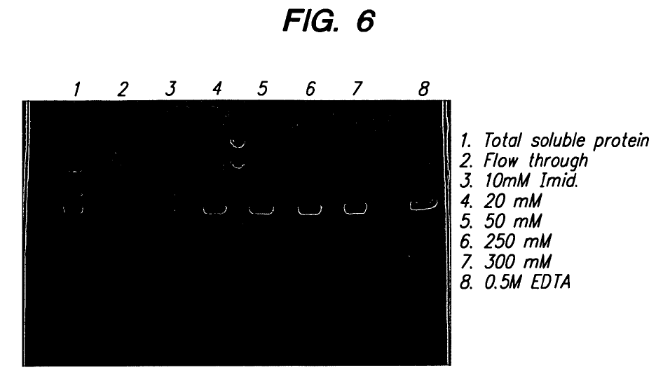

GFP-R5 fusion proteins were purified from cell culture using standard affinity chromatography techniques. The GFP-R5 fusion protein includes a six-residue poly histidine tag. IMAC was used to purify the GFP-R5 fusion protein. Bound proteins were eluted with increasing concentrations of imidazole. The purification results are shown in FIG. 6. Each of the protein-containing fractions glows green under UV light. The total soluble protein fraction, flow through, and the 250 mM imidazole elution fraction all demonstrate the ability to precipitate silica in the purification buffer.

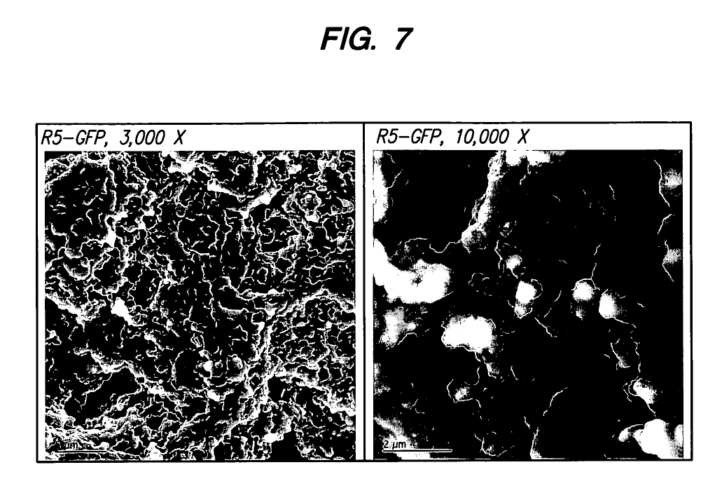

The silica matrices formed with the GFP-R5 fusion protein were examined by SEM. The results are shown in FIG. 7. Based on light microscopy, the morphology of the matrices formed from the R5-GFP fusions was similar to the other matrices produced.

### Example 3

### Silaffin-HRP Fusions

HRP-R5 fusions are made by cloning the nucleotide sequence encoding HRP and the nucleotide sequence encoding R5 in frame with each other into a pET30 expression vector. The nucleotide sequence encoding the fusion protein is:

(SEQ ID NO: 35)

atgcaccatcatcatcatcattcttctggtctggtgccacgcggttctgg
tatgaaagaaaccgctgctgctaaattcgaaTATGAAATACCTGCTGCCG
ACCGCTGCTGCTGGTCTGCTGCTCCTCGCTGCCCAGCCGGCGATGGTATG
AAATACCTGCTGCCGACCGCTGCTGCTGGTCTGCTGCTCCTCGCTGCCCA
GCCGGCCATGGCCATGCAGTTAACCCCTACATTCTACGACAATAGCTGTC
CCAACGTGTCCAACATCGTTCGCGACACAATCGTCAACGAGCTCAGATCC
GATCCCAGGATCGCTGCTTCAATATTACGTCTGCACTTCCATGACTGCTT
TCGTGAATGGTTGCGACGCTAGCATATTACTGGACAACACCACCAGTTTC
CGCACTGAAAAGGATGCATTCGGGAACGCTAACAGCGCCAGGGGCTTTCC
AGTGATCGATCGCATGAAGGCTGCCGTTGAGTCAGCATGCCCACGAACAG
TCAGTTGTGCAGACCTGCTGACTATAGCTGCGCAACAGAGCGTGACTCTT
GCAGGCGGACCGTCCTGGAGAGTGCCGCTCGGTCGACGTGACTCCCTACA
GGCATTCCTAGATCTGGCCAACGCCAACTTGCCTGCTCCATTCTTCACCC
TGCCCCAGCTGAAGGATAGCTTTAGAAACGTGGGTCTGAATCGCTCGAGT
GACCTTGTGGCTCTGTCCGGAGGACACACATTTGGAAAGAACCAGTGTAG
GTTCATCATGGATAGGCTCTACAATTTCAGCAACACTGGGTTACCTGACC
CCACGCTGAACACTACGTATCTCCAGACACTGAGAGGCTTGTGCCCACTG
AATGGCAACCTCAGTGCACTAGTGGACTTTGATCTGCGGACCCCAACCAT
CTTCGATAACAAGTACTATGTGAATCTAGAGGAGCAGAAAGGCCTGATAC
AGAGTGATCAAGAACTGTTTAGCAGTCCAAACGCCACTGACACCATCCCA
CTGGTGAGAAGTTTTGCTAACTCTACTCAAACCTTCTTTAACGCCTTCGT
GGAAGCCATGGACCGTATGGGTAACATTACCCCTCTGACGGGTACCCAAG
GCCAGATTCGTCTGAACTGCAGAGTGGTCAACAGCAACTCTttcgaacgc
cagcacatggacagcccagatctgggtaccgacgacgacgacaaggccat
ggCTtCTTCCTCTAAAAAGTCTGGTTCCTACTCTGGTAGCAAAGGCTCCA
AACGTCGCATCCTG.

The protein sequence of the HRP-R5 fusion protein is as follows:

(SEQ ID NO: 36)

M H H H H H H S S G L V P R G S G M K E T A A A K
F E Y E I P A A D R C C W S A A P R C P A G D G M
K Y L L P T A A A G L L L L A A Q P A M A M Q L T
P T F Y D N S C P N V S N I V R D T I V N E L R S
D P R I A A S I L R L H F H D C F V N G C D A S I
L L D N T T S F R T E K D A F G N A N S A R G F P
V I D R M K A A V E S A C P R T V S C A D L L T I
A A Q Q S V T L A G G P S W R V P L G R R D S L Q
A F L D L A N A N L P A P F F T L P Q L K D S F R
N V G L N R S S D L V A L S G G H T F G K N Q C R
F I M D R L Y N F S N T G L P D P T L N T T Y L Q
T L R G L C P L N G N L S A L V D F D L R T P T I
F D N K Y Y V N L E E Q K G L I Q S D Q E L F S S
P N A T D T I P L V R S F A N S T Q T F F N A F V
E A M D R M G N I T P L T G T Q G Q I R L N C R V
V N S N S F E R Q H M D S P D L G T D D D D K A M
A S S S K K S G S Y S G S K G S K R R I L.

The HRP-R5 fusion protein includes a poly(histidine) tag near the amino terminus. The HRP-R5 fusion protein is purified using immobilized metal affinity chromatography in the same way that recombinant R5 and R5-GFP were isolated.

### Example 4

### Silaffin-Phosphodiesterase Fusions

R5(1)-PDE fusions are made by cloning the nucleotide sequence encoding the enzyme PDE and the nucleotide sequence containing one R5 sequence in frame with each other into a protein expression vector. The nucleotide sequence encoding the fusion protein is:

(SEQ ID NO: 37)

Atgaaagaaaccgctgctgctaaattcgaacgccagcacatggacagccc
agatctgtcctctaaaaagtctggttcctactctggtagcaaaggctcca
aacgtcgcatcctgccagatctgggtaccctggtgccacgcggttccatg
gcgcacaagttcatccacatcacggacattcatcttgtcgagcagggtcg
cgccctctacggccatgaccccggcaaacggttcgagcgctgcatcgaca
gcgtgatcgccgagcacgcggacgcagcgtcttgcgtgatcacgggcgac
ctcgcacatgtcgggcacccggacgcctaccgccagctgtcggagcaatg
cgcgcggttgccaatgccggttcatctgattctcggcaaccacgacagcc
ggaccaacttccgcgagcgcttcccacaggtgccggtggacagcaatggg
ttcgtccagtacgagcaggccatcgggaggttcaggggtctgtttctgga
taccaacgaaccgggaacgcattgcggcgtcttctgcgagcaacgggcaa
actggctttcccagcgcttggcggaggatgattcaccggtgctcctgttc
atgcatcatccggcattccaccttggcatcccggtcatggatcgaatcgg
attggtcgacaacgaatggttgctgacggcgttgaagggccacgagcacc
gcgtcaagcacttgttcttcggccacattcatcgccccatctcgggcagc
tggcgcggcatcccgttctcgacattgcgcggaaccaaccaccaggtggc
gctgcaccttcgggaatcggaagacatcccgggaagcttcgagccaccac
agtacgccgtcgtcctgctcgacgacgattcggtgatcgtgcacctgcat
gactttctcgatcgcagcgagagattctggctaggcgcgtcgagctccgt
cgacaagcttgcggccgcactcgagcaccaccaccaccaccactga.

The protein sequence of the R5(1)-PDE fusion protein is:

(SEQ ID NO: 38)

MKETAAAKFERQHMDSPDLSSKKSGSYSGSKGSKRRILPDLGTLVPRGSM
AHKFIHITDIHLVEQGRALYGHDPGKRFERCIDSVIAEHADAASCVITGD
LAHVGHPDAYRQLSEQCARLPMPVHLILGNHDSRTNFRERFPQVPVDSNG
FVQYEQAIGRFRGLFLDTNEPGTHCGVFCEQRANWLSQRLAEDDSPVLLF
MHHPAFHLGIPVMDRIGLVDNEWLLTALKGHEHRVKHLFFGHIHRPISGSW
RGIPFSTLRGTNHQVALHLRESEDIPGSFEPPQYAVVLLDDDSVIVHLHD
FLDRSERFWLGASSSVDKLAAALEHHHHH.

The R5(1)-PDE fusion protein includes a poly(histidine) tag near the carboxyl terminus. The R5(1)-PDE fusion protein is purified using immobilized metal affinity chromatography in the same way that recombinant R5 and R5-GFP were isolated.

Silica precipitation reaction was carried out using 1 μL, of R5(1)-PDE protein (50 μg/μL), phosphate buffer and hydrolyzed TMOS as described above. Formation of silica spheres occurred within minutes. After immobilization of the enzyme in the spheres, the quantity of enzyme left in the supernatant was found to be about 5 μg. This result indicates that the efficiency of encapsulation was around 90%.

PDE is a phosphodiesterase enzyme capable converting one molecule of bis(4-nitrophenyl)phosphate (BNP) into one molecule of 4-nitrophenyl phosphate and one molecule of p-nitrophenol. FIG. 9 illustrates the enzymatic activity of the PDE protein, the R5(1)-PDE enzyme before autosilification, and the enzymatic activity of the silified matrix after autosilification of the R5(1)-PDE protein. Enzymatic activity, v, is measured in the production of p-nitrophenol, and the production rate is plotted for various concentrations of the substrate, BNP.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A functionalized molecule comprising a parent molecule; and an autosilification moiety, wherein the autosilification moiety is covalently linked to the parent molecule.

2. The functionalized molecule of claim 1, wherein the parent molecule is a polypeptide, a nucleic acid, a lipid, a polysaccharide, an antigen, an antibody, an enzyme, or a drug.

3. The functionalized molecule of claim 1, wherein the parent molecule is an enzyme.

4. The functionalized molecule of claim 1, wherein the parent molecule is an antibody.

5. A silica matrix comprising a functionalized molecule immobilized therein, wherein the functionalized molecule comprises a parent molecule; and an autosilification moiety, wherein the autosilification moiety is covalently linked to the parent molecule.

6. The matrix of claim 5, wherein the matrix is in the form of spheres.

7. The matrix of claim 6, wherein the spheres have an average diameter of from about 10 nm to about 1000 nm.

8. The matrix of claim 5, wherein the matrix is in the form of a sheet.

9. The matrix of claim 5, wherein the matrix is in the form of fibrils.

10. The matrix of claim 5, wherein the matrix is immobilized in a column.

11. A nucleic acid comprising a nucleotide sequence encoding a fusion polypeptide, wherein the fusion polypeptide comprises a parent polypeptide fused in-frame to an autosilification polypeptide.

12. The nucleic acid of claim 11, wherein the parent polypeptide is an enzyme, an antibody, a structural protein, a transmembrane protein, or a synthetic protein.

13. The nucleic acid of claim 11, wherein the nucleotide sequence is operably linked to a promoter.

14. The nucleic acid of claim 13, wherein the promoter is a constitutive promoter or an inducible promoter.

15. A method for producing a product of interest, the method comprising:
contacting a silica matrix with a substrate for an enzyme, wherein the silica matrix comprises a functionalized enzyme immobilized therein, wherein the functionalized enzyme comprises the enzyme; and an autosilification moiety, wherein the autosilification moiety is covalently linked to the enzyme, wherein the functionalized enzyme modifies the substrate and catalyzes production of a product, wherein the product is produced.

16. The method of claim 15, further comprising recovering the product.

17. The method of claim 15, wherein the silica matrix comprises two or more functionalized enzymes in a biosynthetic pathway.

18. The method of claim 15, wherein the product is selected from an isoprenoid, a polyketide, a macrolide, an amino acid, an alkaloid, a synthetic polymer, an antimicrobial agent, and a cancer chemotherapeutic agent.

19. A method of isolating a compound from a sample, the method comprising:
a) contacting a silica matrix with the sample, wherein the silica matrix comprises a functionalized first member of a specific binding pair immobilized therein, wherein the functionalized first member comprises the first member; and an autosilification moiety, wherein the autosilification moiety is covalently linked to the first member, wherein the compound is a second member of the specific binding pair that binds specifically to the first member, and wherein said contacting generates a second member-bound silica matrix; and
b) removing the second member-bound silica matrix from the sample.


SEQUENCE LISTING (39 sequences)

SEQ ID NO: 1 - protein, 19 aa, Artificial Sequence
  Synthetic peptide
SSKKSGSYSGSKGSKRRIL

SEQ ID NO: 2 - protein, 12 aa, Artificial Sequence
  Synthetic peptide
APPGHHHWHIHH

SEQ ID NO: 3 - protein, 12 aa, Artificial Sequence
  Synthetic peptide
KPSHHHHHTGAN

SEQ ID NO: 4 - protein, 12 aa, Artificial Sequence
  Synthetic peptide
MSPHPHPRHHHT

SEQ ID NO: 5 - protein, 12 aa, Artificial Sequence
  Synthetic peptide
MSPHHMHHSHGH

SEQ ID NO: 6 - protein, 12 aa, Artificial Sequence
  Synthetic peptide
LPHHHHLHTKLP

SEQ ID NO: 7 - protein, 12 aa, Artificial Sequence
  Synthetic peptide
APHHHHPHHLSR

SEQ ID NO: 8 - protein, 19 aa, Artificial Sequence
  Synthetic peptide
SSKKSGSYSGSKGSKRRNL

SEQ ID NO: 9 - protein, 22 aa, Artificial Sequence
  Synthetic peptide
SSKKSGSYSGYSTKKSGSRRIL

SEQ ID NO: 10 - protein, 33 aa, Artificial Sequence
  Synthetic peptide
SSKKSGSYYSYGTKKSGSYSGYSTKKSASRRIL

SEQ ID NO: 11 - protein, 5 aa, Artificial Sequence
  Synthetic peptide
VPGXG

SEQ ID NO: 12 - protein, 6 aa, Artificial Sequence
  Synthetic peptide
SGAGAG

SEQ ID NO: 13 - protein, 59 aa, Artificial Sequence
  Synthetic peptide
GAGAGSGAGAGSGAGAGSGAGAGSGAGAGSGAGAGSGAGAGSGAGAGSGAGAGSGAAGY

SEQ ID NO: 14 - protein, 5 aa, Artificial Sequence
  Synthetic peptide
LKPNM

SEQ ID NO: 15 - protein, 4 aa, Artificial Sequence
  Synthetic peptide
KPNM

SEQ ID NO: 16 - protein, 5 aa, Artificial Sequence
  Synthetic peptide
VPGAG

SEQ ID NO: 17 - protein, 5 aa, Artificial Sequence
  Synthetic peptide
VPGSG

SEQ ID NO: 18 - protein, 5 aa, Artificial Sequence
  Synthetic peptide
VPGTG

SEQ ID NO: 19 - protein, 6 aa, Artificial Sequence
  Synthetic linker
AAAGGM

SEQ ID NO: 20 - protein, 14 aa, Artificial Sequence
  Synthetic linker
AAAGGMPPAAAGGM

SEQ ID NO: 21 - protein, 6 aa, Artificial Sequence
  Synthetic linker
AAAGGM

SEQ ID NO: 22 - protein, 8 aa, Artificial Sequence
  Synthetic linker
PPAAAGGM

SEQ ID NO: 23 - protein, 4 aa, Artificial Sequence
  Synthetic linker
IEGR

SEQ ID NO: 24 - protein, 6 aa, Artificial Sequence
  Synthetic linker
GGKGGK

SEQ ID NO: 25 - DNA, 696 bp, Thalassiosira pseudonana
atgaagacttctgccattgcattgcttgccgttctcgccaccactgctgccaccgagccccgccgattgagaactcttgaaggacatgggggagatcactccatctccatgtccatgcacagctcgaaagctgagaagcaagccatcgaggcagctgttgaggaggatgttgctggccctgcaaaggcagccaagcttttcaagcccaaagcaagcaaggctggttccatgcctgatgaggccggtgcaaagagtgccaagatgagcatggacaccaagagtggaaagtcggaggacgcagctgccgtagatgccaaagcttccaaggaatctcacatgtctataagtggtgatatgagcatggccaagtcacacaaggccgaggccgaggacgtcactgagatgtccatggcaaaggccggcaaggatgaggcttcaaccgaggatatgtgtatgcccttcgctaaaagtgacaaggaaatgagcgtcaaatcgaagcaaggaaagaccgagatgagtgtggccgatgccaaggcctcaaaggagtctagcatgccctcttcgaaggctgccaagatcttcaagggaaagagtgggaagtccgggagtctctccatgctcaagagtgaaaaggcaagctccgctcacagcctcagtatgccaaaagctgagaaggtccactccatgagcgcttga

SEQ ID NO: 26 - DNA, 1458 bp, Thalassiosira pseudonana
atgaaagttaccacgtcaatcatcactttgcttttcgcttcttgcggcgctgcagatgtgcagagggtattggaagatgtgaccgagccagcagtaaccacgccagccgccacacctgcacctatcacacccgagccagccacgcctgcacctaccatctgcgaggggcgtaacttctacagggatgatgatacggggaagtgcagtaatgaggccaccggtggaatctatggcactctaatcgaatgttgcgtcgcgatctctggatcagattcctgcccttacgttgatatctgcaatactctccagccgtcaccctctcctgaaaccaacgagccaagtgcaaagcccattaccgctgctcccattagttctgctcctgtcagtgcagctccggtaacatcggctcccgtcgcggccccagttgaaaccacaagtatgacaggcccaactactatcgttgcgagtatcgtttcgacaaatgctccctcttcgacaaatgctccctcttcttccctggaagctgtggtgacgagaattcctgttgaaaccacaaatacggcaagcccaactactaccgctgccagtatcgtttcgacaaacgctccctcgtcttccccagaagctgtggtgacgcctcgtcctacctttcgtccttcccctaagggaaccgagagcaacacgttcccagcttccattgcatctgacgtaatgtttgatccagccagaagcgaaccgaccttcacaccaacctcgagcagccaaccttcatctagcgagcccacgttgagtcctagcgtcagcaaagagccaacgaggtatccaacctccagtccatcgcactcgccgaccaagtctccatcgaagagcccctcttcaagtccaactacctcacccagtgctagtcctaccgagacccccacagagacgcccaccgagtcacctactgagttaccaacactcagtccgactgaatttccctccttatctccgactctttcgcccacatggtctccaacgggctatccaactcttgctccttcgccttcgccttccatttcatcggctccttcggtatcttcagcgccgtcatcttcgccttccatttcatcggctccttcggtatcttcagctccaagtaaaaatttcggtttcctcccgggtcgaaatgaaatgcctactatatcgcccaccgaagaccactacttttttggcaaaagtcacaaatctcacaaatcaaaggcaaccaagaccctcaaggttagcaaatccggaaagagttcgaaaagctccaaaagttcgggcagtaggcctctctttggcgtgtctcaactttcggagggcatcgccgctggatacgccaagagttcaggacgtagtagtcaacaggctgtaggatcgtggatgccggtagctgctgcttgtatccttggtgctttgtccttctttctgaattga

SEQ ID NO: 27 - DNA, 1506 bp, Thalassiosira pseudonana
atgaaagttaccacgtcaatcatcactttgcttttcgcttcttgcggcgctgcagatgtgcagagggtattggaagatgtgaccgagccagcagtaaccacgccagccgccacacctgcacctatcacacccgagccagccacgcctgcaccgaccatctgcgagggacgtaacttctactatgatgaagaaacgaggaagtgcagtaatgaggccaccggtggaatctatggcactctaatagactgctgcgtcgcgatctctggatcagtttcctgcccttacgttgatatctgcaatactctccagccgtcaccctctcctgaaaccaacgagccaagtgcaaagcccattaccgctgctcccattagttctgctcctgtcagtgcagctccggtaacatcggctcccgtcgcagccccagttgaaaccacaagtatgacaggcccaactactatcgttgcgagtatcgtttcgacaaatgctccctctttgacaaatgctccctcttcttccctggaagctgtggtgacgagaattcctgttgaaaccacaaatacggcaagcccaactactaccgctgccagtatcgtttcgacaaatgctccctcttcttccccggaagctgtggtgacgcctcgtcctacatttcgtccttcccctgagggaaccgagagcaacacgtccccagcttccattgcatctgacgtaatgtttggtccacccaaaacctccacaccaacctccacaccaacctcgagcagccacccttcatctagcgagcccacgttgagtcctagcgtcagcaaagagccgacggggtatccaacctccagtccatcgcactcgccgaccaagtctccatcgaagagcccctcttcaagtccaactacctcacccagtgctagtcctactgagacccccacagagacgcccaccgagtcacccaccgagtcacctactgagtcaccgacactcagcccgacggaatctcccacactcagtccgactgaatctccctccttatctccgactctttctaccacatggtctccaacgggctatccaactcttgctccttcgccttcgccttccatttcatcggctccttcggtatcttcagcgccgtcatctccgccttccatttcatcggctccttcggtatcttcagctccaagtaaaaatttcggtttcctcccgggtctaactgaaatgcctactatatcgcccaccgaagaccactacttttttggcaagagtcacaaatctcacaagtctcacaaatcaaaggcaaccaagaccctcaaggttagcaaatccggaaagagtgcgaaaagctccaaaagttcgggcagaaggcctctctttggcgtgtctcaactttcggagggcatcgccgttggatacgccaagagttcaggacgtagtagtcaacaggctgtaggatcgtggatgccggtagctgctgcttgtatccttggtgctttgtccttctttctgaattga

SEQ ID NO: 28 - DNA, 798 bp, Cylindrotheca fusiformis
atgaagctgaccgccatctttccgcttcttttcactgctgtcgggtactgtgctgcccaaagcattgctgacttggcagcagccaatctcagcactgaggattccaagtcagctcagctcatctcagcagactcctcggatgacgcatctgattcctctgtcgagtctgtcgacgccgcctcctctgacgtctctggttcctctgtcgaatctgtcgacgtctctggttcctctctggaatccgttgacgtctctggttcctctctggagtccgtcgacgactccagtgaggactccgaagaggaagaacttcgtatcttgtcttccaagaaatccggatcctactactcctacggcaccaagaagtcgggatcctactcgggatactccacaaagaaatccgcatcgcgtcgcatcttgtcctccaagaaatcgggatcctactcgggatattccacaaagaaatccggatcgcgtcgcatcttgtcctccaagaaatcgggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtccgggggtctcagaggctccatgtaa

SEQ ID NO: 29 - DNA, 60 bp, Artificial Sequence
  Synthetic DNA
tcttcctctaaaaagtctggttcctactctggtagcaaaggctccaaacgtcgcatcctg

SEQ ID NO: 30 - protein, 35 aa, Artificial Sequence
  Synthetic peptide
AEAEAKAKAEAEAKAKSSKKSGSYSGSKGSKRRIL

SEQ ID NO: 31 - protein, 92 aa, Artificial Sequence
  Synthetic peptide
MHHHHHHSSGLVPRGSGMKETAAAKFERQHMDSPDLGTDDDDKAMGYLWIRIRAPSTSLRPHSSTTTTTTEIRLLTKPERKLSWLLPPLSNN

SEQ ID NO: 32 - protein, 65 aa, Artificial Sequence
  Synthetic peptide
MHHHHHHSSGLVPRGSGMKETAAAKFERQHMDSPDLGTDDDDKAMASSKKSGSYSGSKGSKRRIL

SEQ ID NO: 33 - DNA, 915 bp, Artificial Sequence
  Synthetic DNA
atgcaccatcatcatcatcattcttctggtctggtgccacgcggttctggtatgaaagaaaccgctgctgctaaattcgaacgccagcacatggacagcccagatctgggtaccgacgacgacgacaaggccatggcttcttcctctaaaaagtctggttcctactctggtagcaaaggctccaaacgtcgcatcctggccagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataa

SEQ ID NO: 34 - protein, 304 aa, Artificial Sequence
  Synthetic protein
MHHHHHHSSGLVPRGSGMKETAAAKFERQHMDSPDLGTDDDDKAMASSSKKSGSYSGSKGSKRRILASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 35 - DNA, 1263 bp, Artificial Sequence
  Synthetic DNA
atgcaccatcatcatcatcattcttctggtctggtgccacgcggttctggtatgaaagaaaccgctgctgctaaattcgaatatgaaatacctgctgccgaccgctgctgctggtctgctgctcctcgctgcccagccggcgatggtatgaaatacctgctgccgaccgctgctgctggtctgctgctcctcgctgcccagccggcgatggccatgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactctttcgaacgccagcacatggacagcccagatctgggtaccgacgacgacgacaaggccatggcttcttcctctaaaaagtctggttcctactctggtagcaaaggctccaaacgtcgcatcctg

SEQ ID NO: 36 - protein, 421 aa, Artificial Sequence
  Synthetic protein
MHHHHHHSSGLVPRGSGMKETAAAKFEYEIPAADRCCWSAAPRCPAGDGMKYLLPTAAAGLLLLAAQPAMAMQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSFERQHMDSPDLGTDDDDKAMASSSKKSGSYSGSKGSKRRIL

SEQ ID NO: 37 - DNA, 996 bp, Artificial Sequence
  synthetic DNA
atgaaagaaaccgctgctgctaaattcgaacgccagcacatggacagcccagatctgtcctctaaaaagtctggttcctactctggtagcaaaggctccaaacgtcgcatcctgccagatctgggtaccctggtgccacgcggttccatggcgcacaagttcatccacatcacggacattcatcttgtcgagcagggtcgcgccctctacggccatgaccccggcaaacggttcgagcgctgcatcgacagcgtgatcgccgagcacgcggacgcagcgtcttgcgtgatcacgggcgacctcgcacatgtcgggcacccggacgcctaccgccagctgtcggagcaatgcgcgcggttgccaatgccggttcatctgattctcggcaaccacgacagccggaccaacttccgcgagcgcttcccacaggtgccggtggacagcaatgggttcgtccagtacgagcaggccatcgggaggttcaggggtctgtttctggataccaacgaaccgggaacgcattgcggcgtcttctgcgagcaacgggcaaactggctttcccagcgcttggcggaggatgattcaccggtgctcctgttcatgcatcatccggcattccaccttggcatcccggtcatggatcgaatcggattggtcgacaacgaatggttgctgacggcgttgaagggccacgagcaccgcgtcaagcacttgttcttcggccacattcatcgccccatctcgggcagctggcgcggcatcccgttctcgacattgcgcggaaccaaccaccaggtggcgctgcaccttcgggaatcggaagacatcccgggaagcttcgagccaccacagtacgccgtcgtcctgctcgacgacgattcggtgatcgtgcacctgcatgactttctcgatcgcagcgagagattctggctaggcgcgtcgagctccgtcgacaagcttgcggccgcactcgagcaccaccaccaccaccactga

SEQ ID NO: 38 - protein, 330 aa, Artificial Sequence
  recombinant protein
MKETAAAKFERQHMDSPDLSSKKSGSYSGSKGSKRRILPDLGTLVPRGSMAHKFIHITDIHLVEQGRALYGHDPGKRFERCIDSVIAEHADAASCVITGDLAHVGHPDAYRQLSEQCARLPMPVHLILGNHDSRTNFRERFPQVPVDSNGFVQYEQAIGRFRGLFLDTNEPGTHCGVFCEQRANWLSQRLAEDDSPVLLFMHHPAFHLGIPVMDRIGLVDNEWLLTALKGHEHRVKHLFFGHIHRPISGSWRGIPFSTLRGTNHQVALHLRESEDIPGSFEPPQYAVVLLDDDSVIVHLHDFLDRSERFWLGASSSVDKLAAALEHHHHH

SEQ ID NO: 39 - protein, 16 aa, Artificial Sequence
  synthetic peptide
AEAEAKAKAEAEAKAK